(19) United States
(12) Patent Application Publication
(10) Pub. No.: US 2022/0304959 A1
(43) Pub. Date: Sep. 29, 2022

(54) TREATMENT OF LONG HAULERS SYNDROME WITH NICLOSAMIDE

(71) Applicant: First Wave BioPharma, Boca Raton, FL (US)

(72) Inventor: Dinesh Srinivasan, Brooklyn, NY (US)

(21) Appl. No.: 17/703,626

(22) Filed: Mar. 24, 2022

Related U.S. Application Data
(60) Provisional application No. 63/166,696, filed on Mar. 26, 2021.

(51) Int. Cl. A61K 31/167; A61K 9/28; A61K 9/48; A61K 9/00; A61K 45/06; A61P 31/20

(52) U.S. Cl. CPC A61K 31/167; A61K 9/28; A61K 9/4891; A61K 9/0075; A61K 9/0078; A61K 9/0031; A61K 9/0053; A61K 9/2866; A61K 9/284; A61K 45/06; A61P 31/20

(57) ABSTRACT

Disclosed in certain embodiments is a method of treating long-haulers syndrome associated with infection (e.g., Covid-19) comprising administering a therapeutically effective amount of niclosamide or a pharmaceutically acceptable salt thereof to a patient in need thereof.

### BACKGROUND

While most persons with Covid-19 recover and return to normal health, some patients can have symptoms that can last for weeks or even months after recovery from acute illness. This syndrome is referred to as long Covid. Even people who are not hospitalized and who have mild illness can experience persistent or late symptoms. Long Covid is also referred to as chronic Covid, syndrome, Post-Acute Sequelae of SARS-CoV-2 infection (PASC), post Covid syndrome, long term Covid or long-haulers Covid and is characterized by symptoms that persist even after the typical convalescence period of Covid-19. A wide range of symptoms include gastrointestinal, fatigue, headaches, shortness of breath, loss of smell, muscle weakness, low fever and cognitive dysfunction have been reported. Long hauler's syndrome, i.e., persistent symptoms and clinically significant sequelae is also associated with other infections.

There exists a need in the art for a treatment of long-haulers syndrome associated with infections such as Covid-19.

### OBJECTS AND SUMMARY OF THE INVENTION

It is an object of the invention to treat long-haulers syndrome associated with infection comprising administering a therapeutically effective amount of niclosamide or a pharmaceutically acceptable salt thereof to a patient in need thereof.

It is an object of certain embodiments of the invention to treat long-Covid-19 comprising administering a therapeutically effective amount of niclosamide or a pharmaceutically acceptable salt thereof to a patient in need thereof.

It is an object of certain embodiments of the invention to treat post-acute sequelae of Covid-19 (SARS-CoV-2) comprising administering a therapeutically effective amount of niclosamide or a pharmaceutically acceptable salt thereof to a patient in need thereof.

Long haulers are people who have not fully recovered from infection (e.g., COVID-19) weeks or even months after first experiencing symptoms. Some long haulers experience continuous symptoms for weeks or months, while others feel better for weeks, then relapse with old or new symptoms. Patients may also be asymptomatic for the disease. In Covid-19, the syndrome is referred to as long Covid, chronic Covid, syndrome, post Covid syndrome, long term Covid or long-haulers Covid.

### DETAILED DESCRIPTION

Disclosed is a method of treating long-haulers syndrome associated with infection (e.g., Covid-19) comprising administering a therapeutically effective amount of niclosamide or a pharmaceutically acceptable salt thereof to a patient in need thereof. In certain embodiments, the infection is viral or bacterial (e.g., Lyme's disease). In certain embodiments, the viral infection is selected from Covid-19 (SARS-CoV-2), chinkungunya, dengue, SARS and MERS. In certain embodiments, the patient is treated for post-acute sequelae of Covid-19 (SARS-CoV-2).

In certain embodiments, the administration treats symptoms associated with one or more organ systems selected from gastrointestinal, pulmonary, renal, hepatic, dermatologic, coronary and nervous.

In certain embodiments, the symptoms of the organ systems are associated with one or more organs selected from intestine, lungs, heart and brain.

In certain embodiments, the treatment provides one or both of an anti-inflammatory response and an antiviral response.

In certain embodiments, the administration is in an effective amount to cause an antiviral response by induction of autophagy.

In certain embodiments, the administration is in an effective amount to cause an anti-inflammatory response by one or more of mitigation of pathogenic lamina propria T cells or reduction of pro-inflammatory cytokines.

In certain embodiments, the administration changes the microflora of the intestine.

In certain embodiments, the administration regulates the presence of an organism selected from one or more of pseudomonas, Clostridium difficile, Bacteroides uniformis, Enterococcus faecalis, monoglobus pectinilyticus, porphyromonasendodontalis, veillonella tobetsuensis, Bifidobacterium breve, ruminococcus gnavus, ruminococcus torques, bacteroides dorei, ruminococcus gnavus, chikungunya virus.

In certain embodiments, the treatment further comprises changing the administration in response to the changes of the microflora of the intestine. In certain embodiments, the changes in microflora is observed by fecal analysis. In certain embodiments, the change in administration is a reduced dose, an increased dose or a cessation of treatment.

In certain embodiments, the treatment further comprises changing the administration in response to the changes of the microflora of the intestine or nasopharyngeal cavity. In certain embodiments, the changes in microflora is observed by fecal analysis or nasal swab analysis. In certain embodiments, the change in administration is a reduced dose, an increased dose or a cessation of treatment.

In certain embodiments, the administration is by a route selected from oral, nasal, inhalation, rectal, topical, parenteral or transdermal.

In certain embodiments, the administration is by inhalation such as by dry powder, aerosols, nebulization or intubation.

In certain embodiments, the administration is rectal, such as by enema or suppository.

In certain embodiments, the administration is oral. In certain oral embodiments, the daily dose does not exceed about 2000 mg. in other embodiments, the daily dose is from about 200 mg to about 1800 mg, from about 400 mg to about 1600 mg, from about 600 mg to about 1400 mg, from about 800 mg to about 1200 mg, or from about 1000 mg to about 1400 mg. In certain embodiments, the daily dose is administered once daily or can be administered in divided doses, e.g., twice daily, thrice daily, four times daily, 5 times daily or 6 times daily.

In certain embodiments, the niclosamide or pharmaceutically acceptable salt thereof is administered 400 mg three times daily.

In certain embodiments, the oral administration can be with an oral solid dosage form. e.g., a tablet or capsule. In certain embodiments, the oral solid dosage form can include an enteric coating, e.g., an acrylic polymer or a cellulosic phthalate.

In certain embodiments, the oral administration is with an immediate release dosage form, a controlled release dosage form, a delayed release dosage form, a sustained release dosage form, a pulsatile dosage form or a chronotherapeutic dosage form.

In certain embodiments, the administration is with food. In other embodiments, the administration is in the fasted state.

In certain embodiments, the administration treats intestinal symptoms.

In certain embodiments, the treatment results in a reduction or elimination of diarrhea. The reduction can be, e.g., one or both of frequency of diarrhea or severity of diarrhea. In certain embodiments, the intestinal symptoms are associated with post-acute sequalae.

In certain embodiments herein, the patient is asymptomatic of infection (e.g., Covid-19). In certain embodiments, the patient is asymptomatic at the first administration. In certain embodiments, the patient was asymptomatic of infection (e.g., Covid-19) at all times prior to first administration. In certain embodiments, the patient was symptomatic of Covid-19 at a time prior to first administration. In certain embodiments, the patient is symptomatic of infection (e.g., Covid-19) at first administration and the treatment is continued when the patient is asymptomatic. In certain embodiments, the asymptomatic patient has a negative infection diagnosis (e.g., a Covid-19 rectal swab or PCR test).

In certain embodiments, the niclosamide or pharmaceutically acceptable salt thereof is micronized. The niclosamide or pharmaceutically acceptable salt can have, e.g., a D90 from about 1 micron to about 20 microns, from about 2 microns to about 15 microns, from about 3 microns to about 12 microns, from about 4 microns to about 10 microns or from about 5 microns to about 9 microns.

In certain embodiments, the administration results in a concentration of niclosamide i in the intestine (e.g., surface tissue) which is more than the systemic plasma concentration. In certain embodiments, the ratio of the concentration of niclosamide in the intestine to the systemic plasma is greater than 2, great than 5, greater than 10, greater than 25 greater than 50, greater than 75, greater than 100, greater than 150, greater than 200, greater than 250, greater than 300, or greater than 350.

In certain embodiments, the treatment further comprises administering an anti-viral agent or antibiotic by the same route or a different route of administration than the niclosamide or pharmaceutically acceptable salt thereof. In certain embodiments, the anti-viral agent is a viral mimetic, a nucleotide analog, a nucleotide mimic, a sialidase inhibitor, or a protease inhibitor. In certain embodiments, the anti-viral agent is amantadine, rimantadine, ribavirin, zanamivir, oseltamivir, remdesivir or a pharmaceutically acceptable salt thereof.

In certain embodiments, the treatment further comprises administering an additional active agent to treat intestinal symptoms selected from the group consisting of aminosalicylate, antibiotics, biologics, corticosteroids, immunomodulators, anti-diarrheals. In certain embodiments, the treatment further comprises administering infliximab.

In certain embodiments, the treatment further comprises measuring the microflora in the intestine of the patient. In certain embodiments, the measuring is performed before the first administration. In certain embodiments, the measuring is after the first administration. In certain embodiments, the treatment comprises continuing treatment until a subsequent measuring indicates improvement in the microflora.

In certain embodiments, the administration treats one or more symptoms selected from fatigue, shortness of breath, cough, joint pain, chest pain, difficulty with thinking and concentration (brain fog), depression, muscle pain, headache, intermittent fever, heart palpitations, inflammation of the heart muscle, lung function abnormalities, acute kidney injury, rash, hair loss, smell and taste problems, sleep issues, difficulty with concentration, memory problems, depression, anxiety and change in mood.

In certain embodiments, the methods of the present invention provide prophylaxis of long hauler's syndrome associated with infection (e.g., Covid-19). The prophylactic treatment can be administered to a patient exhibiting signs of or diagnosed with infection (e.g., Covid-19) in order to avoid or lessen the symptoms of long hauler's syndrome. In other embodiments, the prophylactic treatment can be administered to a patient that was exposed to or potentially exposed to infection (e.g., Covid-19) in order to avoid or lessen the symptoms of the active disease or of long hauler's syndrome.

In certain embodiments, the niclosamide compounds (or pharmaceutically acceptable salts and/or co-crystals thereof, e.g., niclosamide), have one or more properties that include, but are not limited to: a particular purity (e.g., a chemical purity of greater than about 99.0%) or a particular particle size (e.g., a particular particle size distribution and/or a particular particle size range and/or a specific surface area range). In an aspect, the niclosamide compounds described herein (e.g., niclosamide) can form part of compositions, dosage forms (e.g., solid dosage forms; e.g., unit dosage forms; e.g., unit solid dosage forms), and the like, which are suitable for respiratory administration (e.g., inhalation and/or intranasally). In another aspect, the niclosamide compounds described herein (e.g., niclosamide) can form part of compositions, dosage forms (e.g., solid dosage forms; e.g., unit dosage forms; e.g., unit solid dosage forms), and the like, which are suitable for administration to the GI tract (e.g., orally, such as via tablet or pill; or rectally such as via enema)). This disclosure also features methods of making and using the same.

Methods of Treatment

In one aspect, provided herein is a method for treating long hauler's syndrome in a subject in need thereof, the method comprising administering an effective amount of a niclosamide compound, or a pharmaceutically acceptable salt thereof, to the subject. In some embodiments, the long hauler's syndrome is severe or mild.

In some embodiments of the methods herein, the subject exhibits one or more symptoms selected from the group consisting of fever, cough, fatigue, shortness of breath (dyspnea), muscle and/or joint pain, sore throat, headache, conjunctivitis, diarrhea, lack or loss of appetite, vomiting, abdominal pain, thrombosis, loss of taste, loss of smell, and/or chills. In certain of these embodiments, the subject exhibits one or more symptoms selected from the group consisting of fever, cough, and shortness of breath. In some embodiments, the subject exhibits one or more digestive symptoms (e.g., extra-pulmonary symptoms) selected from the group consisting of diarrhea, lack or loss of appetite, vomiting, and abdominal pain. In some embodiments, the subject exhibits diarrhea. In some embodiments, the subject has a rash and/or a lesion on one or more toes. In some embodiments, the subject exhibits thrombosis. In some embodiments, the subject has cytokine storm syndrome.

Also provided herein is a method for treating thrombosis in a subject having long hauler's syndrome, the method comprising administering an effective amount of a niclosamide compound, or a pharmaceutically acceptable salt thereof, to the subject. In some embodiments, the method further includes administering an anticoagulation agent (e.g., any of the anticoagulation agents described herein). In some embodiments, a subject having thrombosis has a D-dimer level of at least about 2 μg/mL. For example, at least about 2.5 μg/mL, at least about 3 μg/mL, at least about 3.5 μg/mL, at least about 4 μg/mL, at least about 4.5 μg/mL, or at least 5 μg/mL. In some embodiments, a subject with thrombosis has deep vein thrombosis (DVT), pulmonary embolism (PE), femoral vein thrombosis, myocardial infarction, superior vena cava thrombosis, jugular vein thrombosis, stroke, cerebral venous sinus thrombosis, cavernous sinus thrombosis, retinal vein occlusion, portal vein thrombosis, Budd-Chiari syndrome, renal vein thrombosis, or a combination thereof.

Also provided herein is a method for treating cytokine storm syndrome in a subject having long hauler's syndrome, the method comprising administering an effective amount of a niclosamide compound, or a pharmaceutically acceptable salt thereof, to the subject. In some embodiments, the method further includes administering an IL-6 targeted therapy (e.g., any of the IL-6 targeted therapies described herein). In some embodiments, a subject has an IL-6 level of at least about 80 pg/mL. For example, at least about 85 pg/mL, at least 90 pg/mL, at least 95 pg/mL, at least 100 pg/mL, at least 105 pg/mL, at least 110 pg/mL, or at least 115 pg/mL.

Long hauler's syndrome associated with COVID-19) may cause digestive symptoms for several reasons. For example, SARS-CoV-2 can invade the human body by binding to the human angiotensin converting enzyme 2 (ACE-2) receptor, which can cause liver tissue injury; SARS-CoV-2 can indirectly or directly damage the digestive system through an inflammatory response; SARS-CoV-2 may cause disorders of the intestinal flora. Changes in the composition and function of the digestive tract flora, e.g., by niclosamide, can affect the respiratory tract through the common mucosal immune system, and respiratory tract flora disorders can also affect the digestive tract through immune regulation (e.g., SARS-CoV-2 may affect the gut-lung axis). See, e.g., Pan et al. Clinical Characteristics of COVID-19 Patients with Digestive Symptoms in Hubei, China: A Descriptive, Cross-Sectional, Multicenter Study. Am. J. Gastroenterol 2020 Mar. 19; [EPub Ahead of Print]. Accordingly, also provided herein is a method for treating one or more digestive symptoms in a subject having long hauler's syndrome, the method comprising administering an effective amount of a niclosamide compound, or a pharmaceutically acceptable salt thereof, to the subject.

In certain embodiments of the methods described herein, the subject does not exhibit an accompanying respiratory symptom. In certain other embodiments, the subject exhibits an accompanying respiratory symptom.

The methods disclosed herein can further comprise a step of identifying a subject having long hauler's syndrome associated with COVID-19. Identification of a subject as having long hauler's syndrome associated with COVID-19 can include the detection of RNA from severe acute respiratory syndrome coronavirus 2 (SARS-CoV-2) in a biological sample from the subject. In some embodiments, the biological sample is a respiratory sample. Non-limiting examples of respiratory samples that can be used to detect SARS-CoV-2 include a nasopharyngeal swab sample, an oropharyngeal swab sample, a sputum sample, a bronchoalveolar lavage sample, a nasopharyngeal aspirate, a nasopharyngeal wash, a nasal aspirate, a nasal wash, and a lower respiratory tract aspirate. In some embodiments, the biological sample is a fecal sample and/or an anal/rectal swab sample. In some embodiments, polymerase chain reaction (PCR) is used to detect RNA from SARS-CoV-2 in a sample from a subject. Non-limiting examples of types of PCR that can be used to identify a subject as having COVID-19 include reverse transcription PCR (RT-PCR), real-time PCR (e.g., quantitative PCR (qPCR)), and real-time RT-PCR (rRT-PCR). In some embodiments, a specific gene from SARS-CoV-2 is detected. For example, the E gene, RNA-dependent RNA polymerase gene (RdRp) gene, ORF1a gene, ORF1b gene, N gene, or a combination thereof can be detected using primers or probes specific to the gene or a portion thereof. In some embodiments, detection of RNA from SARS-CoV-2 can include using a kit comprising, for example, PCR reagents and primers and/or probes for detecting RNA from SARS-CoV-2 (e.g., primers and/or probes specific to the E gene, RNA-dependent RNA polymerase gene (RdRp) gene, ORF1a gene, ORF1b gene, N gene, or a combination thereof). Many commercial kits are available to detect RNA from SARS-CoV-2. Non-limiting examples of such kits include PowerChek™ 2019-nCov RT-PCR kit (Kogene Biotech), RT-PCR Allplex 2019-nCoV Assay (Seegene); STANDARD M n-CoV Real-Time Detection Kit (SD Biosensor); rRT-PCR XPERT® Xpress SARS-CoV-2 (Cepheid Innovation); and Primerdesign Ltd COVID-19 GENESIG® Real-Time PCR assay. See also, the kits approved by the Food and Drug Administration (fda.gov/medical-devices/emergency-situations-medical-devices/emergency-use-authorizations#covid19ivd).

In some embodiments, the E gene and RdRp gene specific to SARS-CoV-2 is detected (see, e.g., PowerChek™ 2019-nCov RT-PCR kit; RT-PCR Allplex 2019-nCoV Assay; and STANDARD M n-CoV Real-Time Detection Kit (SD Biosensor)). In some embodiments, the ORF1a gene and N gene are detected (see, e.g., DiaPlexQ™ Novel Coronavirus Detection Kit (2019-nCoV) (SolGent Co., Ltd.)) In some embodiments, the RdRP gene, E gene, and N gene are detected (see, e.g., Corman et al. Eurosurveillance, 25, 2000045 (2020)). In some embodiments, the ORF1 ab genome region is detected (see, e.g., Primerdesign Ltd COVID-19 GENESIG® Real-Time PCR assay). In some embodiments, the N2 gene and E gene are detected (see, e.g., rRT-PCR XPERT® Xpress SARS-CoV-2 (Cepheid Innovation)). In some embodiments, primers and probes to detect the RdRp gene spanning nucleotides 12621-12727 and 14010-14116 (positions according SARS-CoV, NC_004718) can be used to detect SARS-CoV-2. See, e.g. the World Health Organization Protocol from the National Reference Center for Respiratory Viruses, Institut Pasteur, Paris (www.who.int/docs/default-source/coronaviruse/real-time-rt-per-assays-for-the-detection-of-sars-cov-2-institut-pasteur-paris.pdf?sfvrsn=3662fcb6_2). In some embodiments, a first gene is detected in a screening test and a second gene is detected for confirmation. For example, the N gene from SARS-CoV-2 can be detected in a screening assay and ORF1b from SARS-CoV-2 can be detected as a confirmatory assay.

In some embodiments, rRT-PCR can be used to monitor a subject with long hauler's syndrome associated with COVID-19. For example, prior to starting a therapy as described herein (e.g., a niclosamide compound, or a pharmaceutically acceptable salt thereof as described herein), a biological sample can be obtained from the subject and the level of SARS-CoV-2 RNA (e.g., the level of RNA corresponding to a SARS-CoV-2 gene described herein) determined in the biological sample. This sample can be considered a base-line sample. The subject can then be administered one or more doses of a therapy as described herein (e.g., a niclosamide compound, or a pharmaceutically acceptable salt thereof as described herein) and the levels of SARS-CoV-2 RNA can be monitored (e.g., after the first dose, second dose, third dose, etc. or after one week, two weeks, three weeks, four weeks, etc.). If the level of SARS-CoV-2 RNA is lower than the baseline sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction etc.), this is indicative of responsiveness to therapy. In some embodiments, the level of SARS-CoV-2 RNA in a biological sample obtained from the subject (n) is compared to the sample taken just previous (n−1). If the level of SARS-CoV-2 RNA in the n sample is lower than the n−1 sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction, e.g., greater than 50% reduction, greater than 60% reduction, greater than 70% reduction, greater than 75% reduction, greater than 80% reduction, greater than 85% reduction, greater than 90% reduction, greater than 95% reduction, greater than 98% reduction, greater than 99% reduction, etc.), this is indicative of responsiveness to therapy. In the case of responsiveness to therapy, the subject can be administered one or more doses of therapy (e.g., a niclosamide compound, or a pharmaceutically acceptable salt thereof) and the SARS-CoV-2 RNA can be continued to be monitored.

In certain embodiments, the reduction is a 5% reduction, 10% reduction, 15% reduction, 20% reduction, 25% reduction, 30% reduction, 35% reduction, 40% reduction, 45% reduction, 50% reduction, 55% reduction, 60% reduction, 65% reduction, 70% reduction, 75% reduction, 80% reduction, 85% reduction, 90% reduction, 95% reduction, 98% reduction, or 99% reduction.

In some embodiments, also provided herein are methods for reducing the viral load of SARS-CoV-2 in a subject with long hauler's syndrome associated with COVID-19, the method comprising administering a niclosamide compound, or a pharmaceutically acceptable salt thereof, to a subject having long hauler's syndrome associated with COVID-19. In some embodiments, the viral load is reduced in one or more of: bronchoalveolar lavage (BAL) fluid (BALF), saliva, bronchial fluid (BF), cerebrospinal fluid (CSF), urine, sputum, stool, saliva, mucus, phlegm, blood, serum, skin, and fingernail (e.g., stool) from the subject. In some embodiments, the saliva is parotid saliva (PS). In certain embodiments, the methods described herein reduce intestinal viral load, e.g., as measured as described herein by measuring the number of viral copies in a stool sample from the subject.

In some embodiments, RNA from SARS-CoV-2 is detected in a sample from the subject to determine viral load. In some embodiments, the sample comprises bronchoalveolar lavage (BAL) fluid (BALF), saliva, bronchial fluid (BF), cerebrospinal fluid (CSF), urine, sputum, stool, saliva, mucus, phlegm, blood, serum, skin, or fingernail (e.g., stool) from the subject.

In some embodiments, administration of a niclosamide compound (e.g., niclosamide) causes a reduction in time to fecal (SARS-CoV-2) RNA viral clearance (e.g. 50% clearance, 55% clearance, 60% clearance, 65% clearance, 70% clearance, 75% clearance, 80% clearance, 85% clearance, 90% clearance, 95% clearance, 98% clearance, or 99% clearance; e.g., 50% clearance) in a subject relative to administration of a placebo (e.g., the time to fecal (SARS-CoV-2) RNA viral clearance in a subject receiving a niclosamide compound (e.g., niclosamide) is two times shorter or three times shorter or five times shorter, or ten times shorter or more relative to administration of a placebo).

In some embodiments, administration of a niclosamide compound (e.g., niclosamide) causes an increase in the rate of fecal (SARS-CoV-2) RNA viral clearance (e.g. 50% clearance, 55% clearance, 60% clearance, 65% clearance, 70% clearance, 75% clearance, 80% clearance, 85% clearance, 90% clearance, 95% clearance, 98% clearance, or 99% clearance; e.g., 50% clearance) in a subject over a particular time period (e.g., 1 day, 2 days, 3 days, 4 days, 5 days, 6 days, 7 days, 8, days, 9 days, 10 days, 11 days, 12 days, 13 days, or 14 days or 21 days or 28 days or 35 days or 42 days; e.g., 14 days) relative to administration of a placebo (e.g., the rate of fecal (SARS-CoV-2) RNA viral clearance in a subject receiving a niclosamide compound (e.g., niclosamide) over a particular time period (e.g., 1-14 days) is two times greater or three times greater or five times greater, or ten times greater or more relative to administration of a placebo).

In some embodiments, a number of subjects receiving a niclosamide compound (e.g., niclosamide) and achieving a particular fecal (SARS-CoV-2) RNA viral clearance (e.g. 50% clearance, 55% clearance, 60% clearance, 65% clearance, 70% clearance, 75% clearance, 80% clearance, 85% clearance, 90% clearance, 95% clearance, 98% clearance, or 99% clearance; e.g., 50% clearance) over a particular time period (e.g., 1 day, 2 days, 3 days, 4 days, 5 days, 6 days, 7 days, 8, days, 9 days, 10 days, 11 days, 12 days, 13 days, or 14 days; e.g., 14 days or 21 days or 28 days or 35 days or 42 days; e.g., 14 days) is greater than a number of subjects receiving a placebo over the same time period (e.g., twice as many subjects, three times as many subjects twice as many subjects, three times as many subjects)

In some embodiments, administration of a niclosamide compound (e.g., niclosamide) causes a reduction in time from first dose of a niclosamide compound (e.g., niclosamide) to the first formed stool (this formed stool must have been followed by a non-watery stool) or time from the first dose of niclosamide to the last watery stool in a subject relative to administration of a placebo (e.g., two times shorter or three times shorter or five times shorter, or ten times shorter or more relative to administration of a placebo).

In some embodiments of the methods described herein, the subject is a subject at risk. For example, the subject is at risk of being infected with SARS-CoV-2 and developing long hauler's syndrome associated with COVID-19. In certain embodiments, the subject is 60 years of age or older. In certain embodiments, the subject suffers from one or more preexisting medical conditions selected from the group consisting of lung disease, cardiovascular disease, and diabetes.

In some embodiments, provided herein are methods of preventing long hauler's syndrome associated with COVID-19 infection in a subject in need thereof, the method comprising administering an effective amount (e.g., a prophylactically effective amount) of a niclosamide compound, or a pharmaceutically acceptable salt thereof, to the subject. In some embodiments, the subject is at risk of developing long hauler's syndrome associated with COVID-19. For example, in some embodiments, the subject is at risk of being infected with SARS-CoV and developing long hauler's syndrome associated with COVID-19.

In another aspect, provided herein is a method of reducing the risk of developing long hauler's syndrome associated with COVID-19 in a subject at risk thereof, the method comprising administering an effective amount (e.g., a prophylactically effective amount) of a niclosamide compound, or a pharmaceutically acceptable salt thereof, to the subject.

In certain embodiments, the subject at risk of developing long hauler's syndrome associated with COVID-19 is a healthcare worker (e.g., emergency room physician or nurse, first responder).

In certain embodiments, the subject at risk of developing long hauler's syndrome associated with COVID-19 is 60 years of age or older.

In certain embodiments, the subject at risk of developing long hauler's syndrome associated with COVID-19 suffers from one or more preexisting medical conditions selected from the group consisting of lung disease, cardiovascular disease, and diabetes.

In certain embodiments, the subject at risk of developing long hauler's syndrome associated with COVID-19 is a resident of an assisted living facility or nursing home, a patient in a hospital for an unrelated treatment (i.e., not related to treatment for long hauler's syndrome associated with COVID-19), or a person incarcerated or working in a prison or jail setting.

In certain embodiments, the subject at risk of developing long hauler's syndrome associated with COVID-19 is unresponsive to treatment with remdesivir.

In certain embodiments, the subject at risk of developing long hauler's syndrome associated with COVID-19 has been exposed to the virus or presumed to have been exposed to the virus.

In certain embodiments, the compound is administered prior to exposure to the virus or prior to presumed exposure to the virus (e.g., prior to contact with one or more individuals having or presumed to have COVID-19 and/or prior to contact with one or more articles contaminated with the virus). For example, the compound can be administered immediately after or shortly after exposure or presumed exposure to the virus.

In another aspect, the disclosure features a method of reducing the risk of developing long hauler's syndrome associated with COVID-19 in a subject (e.g., a human) at risk thereof, the method comprising administering an effective amount of niclosamide:

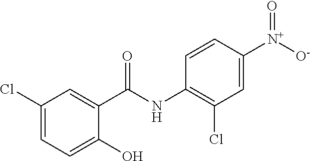 [SMILES: O=C(Nc1ccc([N+](=O)[O-])cc1Cl)c1cc(Cl)ccc1O]

or a pharmaceutically acceptable salt thereof, to the subject (e.g., the human).

In certain of these embodiments, the subject is selected from the group consisting of a healthcare worker, a resident of an assisted living facility or nursing home, a patient in a hospital for an unrelated treatment, and a person incarcerated or working in a prison or jail setting. In certain embodiments, the subject is 60 years of age or older. In certain embodiments, the subject suffers from one or more preexisting medical conditions selected from the group consisting of lung disease, cardiovascular disease, cancer, colitis, and an endocrine disease. In certain embodiments, the compound is administered prior to exposure to the coronavirus or immediately after exposure or presumed exposure to the coronavirus.

In some embodiments, the methods described herein further comprise one or more of the following: quarantine, self-quarantine, social distancing, frequent handwashing, and frequent environmental sanitization.

In some embodiments of the methods described herein, the subject is a human.

In some embodiments of the methods described herein, the effective amount of the niclosamide compound, or a pharmaceutically acceptable salt thereof, can be systemically administered to the subject. In some embodiments of the methods described herein, the effective amount of the niclosamide compound, or a pharmaceutically acceptable salt thereof, can be administered to one or more locations in the subject. For example, in some embodiments, the niclosamide compound, or a pharmaceutically acceptable salt thereof, can be administered to one or more of: the respiratory system, the GI tract, and the skin of the subject. In some embodiments, the niclosamide compound, or a pharmaceutically acceptable salt thereof, can be administered to one or more of: the lungs, intestine, and skin. In some embodiments, the niclosamide compound, or a pharmaceutically acceptable salt thereof, is administered to epithelial tissue of the subject. For example, the niclosamide compound, or a pharmaceutically acceptable salt thereof, is administered to the epithelial tissue of the lungs, blood vessels, heart, GI tract, or a combination thereof. In some embodiments, the niclosamide compound, or a pharmaceutically acceptable salt thereof, is administered to endothelial cells in the subject. In some embodiments of the methods described herein, the effective amount of the niclosamide compound, or a pharmaceutically acceptable salt thereof, can be locally and/or topically administered to the subject.

In some embodiments, the methods described herein comprise administering the effective amount of the niclosamide compound, or a pharmaceutically acceptable salt thereof, to the respiratory system of the subject. In certain embodiments, the methods described herein comprise locally administering an effective amount of the niclosamide compound, or a pharmaceutically acceptable salt thereof, to the respiratory system of the subject. In certain embodiments, the methods described herein comprise topically administering an effective amount of the niclosamide compound, or a pharmaceutically acceptable salt thereof, to the respiratory system of the subject. In certain of the foregoing embodiments, the methods described herein comprise locally and topically administering an effective amount of the niclosamide compound, or a pharmaceutically acceptable salt thereof, to the respiratory system of the subject.

In some embodiments, the methods described herein comprise administering the effective amount of the niclosamide compound, or a pharmaceutically acceptable salt thereof, to the lungs of the subject. In certain embodiments, the methods described herein comprise locally administering an effective amount of the niclosamide compound, or a pharmaceutically acceptable salt thereof, to the lungs of the subject. In certain embodiments, the methods described herein comprise topically administering an effective amount of the niclosamide compound, or a pharmaceutically acceptable salt thereof, to the lungs of the subject. In certain embodiments, the methods described herein comprise locally and topically administering an effective amount of the niclosamide compound, or a pharmaceutically acceptable salt thereof, to the lungs of the subject.

In certain embodiments of the methods described herein, the niclosamide compound, or a pharmaceutically acceptable salt thereof, is administered by inhalation.

In another aspect, provided herein is a method for treating long hauler's syndrome associated with COVID-19 in a subject in need thereof, the method comprising administering an effective amount of a niclosamide compound, or a pharmaceutically acceptable salt thereof, to the GI tract of the subject. In certain embodiments, the method comprises locally administering an effective amount of the niclosamide compound, or a pharmaceutically acceptable salt thereof to the GI tract of the subject. In certain embodiments, the method comprises topically administering an effective amount of the niclosamide compound, or a pharmaceutically acceptable salt thereof to the GI tract of the subject.

In certain of these embodiments, the niclosamide compound, or a pharmaceutically acceptable salt thereof, is administered by rectal administration. As a non-limiting example of the foregoing embodiments, the niclosamide compound, or a pharmaceutically acceptable salt thereof, is administered by enema, rectal gel, rectal foam, rectal aerosol, or suppository. For example, the niclosamide compound, or a pharmaceutically acceptable salt thereof, can be administered by enema.

In some embodiments, the method comprises orally administering the niclosamide compound, or a pharmaceutically acceptable salt thereof, as a pharmaceutical composition, wherein the pharmaceutical composition is capable of local delivery to the digestive or GI tract. In certain of these embodiments, the pharmaceutical composition comprises one or more pharmaceutically acceptable excipients that chemically and/or structurally predispose the composition for delivery of the niclosamide compound, or a pharmaceutically acceptable salt thereof, to the lower GI tract. For example, the composition comprises one or more pharmaceutically acceptable excipients that chemically and/or structurally predispose the composition for delivery of the niclosamide compound, or a pharmaceutically acceptable salt thereof, to the colon. As another non-limiting example, the composition comprises one or more pharmaceutically acceptable excipients that chemically and/or structurally predispose the composition for delivery of the niclosamide, or a pharmaceutically acceptable salt thereof, to the ascending colon and/or transverse colon and/or distal colon. As another non-limiting example, the composition comprises one or more pharmaceutically acceptable excipients that chemically and/or structurally predispose the composition for delivery of niclosamide, or a pharmaceutically acceptable salt thereof, to the small intestine (e.g., to the ileum). In some embodiments, the composition is a solid dosage form (e.g., a solid unit dosage form), such as a tablet or pill.

In another aspect, provided herein is a method for treating long hauler's syndrome associated with COVID-19 in a subject in need thereof, the method comprising orally administering an effective amount of niclosamide:

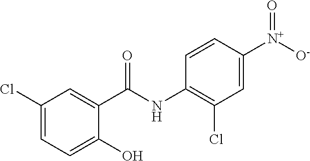 [SMILES: O=C(Nc1ccc([N+](=O)[O-])cc1Cl)c1cc(Cl)ccc1O]

or a pharmaceutically acceptable salt thereof, to the subject.

In certain of these embodiments, the method comprises administering niclosamide.

In certain embodiments, subject exhibits a digestive symptom. In certain embodiments, the subject exhibits a symptom selected from the group consisting of a lack or loss of appetite, diarrhea, vomiting, abdominal pain, a digestive disease, and combinations thereof. In certain embodiments, the subject exhibits a symptom selected from the group consisting of lack or loss of appetite, diarrhea, vomiting, abdominal pain, and combinations thereof. As a non-limiting example of the foregoing embodiments, the subject exhibits diarrhea.

In certain embodiments, the subject does not exhibit an accompanying respiratory symptom. In certain other embodiments, the subject exhibits an accompanying respiratory symptom.

In certain embodiments, the subject suffers from one or more preexisting medical conditions selected from the group consisting of lung disease, cardiovascular disease, cancer, hypertension, and an endocrine disease.

In certain embodiments, the subject suffers from, or is predisposed to suffer from, colitis.

In certain embodiments, the colitis is an autoimmune colitis. In certain embodiments, the colitis is an inflammatory bowel disease. In certain embodiments, the colitis is ulcerative colitis or Crohn's disease. In certain embodiments, the colitis is iatrogenic autoimmune colitis. In certain embodiments, the colitis is selected from the group consisting of colitis induced by treatment with adoptive cell therapy, colitis associated by one or more alloimmune diseases, collagenous colitis, lymphocytic colitis, and microscopic colitis.

In certain embodiments, the method further comprises administering a second therapeutic agent. As a non-limiting example of the foregoing embodiments, the second therapeutic agent is selected from the group consisting of azithromycin, remdesivir, hydroxychloroquine, and chloroquine.

In certain embodiments, the niclosamide, or a pharmaceutically acceptable salt thereof, is administered by tablet or pill.

In certain embodiments, the method comprises orally administering the niclosamide, or a pharmaceutically acceptable salt thereof, as a pharmaceutical composition, wherein the pharmaceutical composition is capable of local delivery to the lower GI tract.

In certain embodiments, the method comprises orally administering the niclosamide, or a pharmaceutically acceptable salt thereof, as a pharmaceutical composition, wherein the pharmaceutical composition is capable of local delivery to the colon.

In certain embodiments, the method comprises orally administering the niclosamide, or a pharmaceutically acceptable salt thereof, as a pharmaceutical composition, wherein the pharmaceutical composition is capable of local delivery to the small intestine.

In some embodiments, long hauler's syndrome associated with COVID-19 is severe or mild. Also provided herein are methods for treating mild long hauler's syndrome associated with COVID-19 in a subject in need thereof, the method comprising administering an effective amount of a niclosamide compound, or a pharmaceutically acceptable salt thereof, to the subject. In some embodiments, a subject having mild COVID-19 has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, a subject having mild long hauler's syndrome associated with COVID-19 does not have a severe disease complication. Severe disease complications can include, but are not limited to, respiratory failure, requirement of mechanical ventilation, septic shock, and non-respiratory organ failure. In some embodiments, a subject having mild long hauler's syndrome associated with COVID-19 does not have digestive symptoms. For example, the subject does not have diarrhea, abdominal pain, or vomiting. In some embodiments, a subject having mild long hauler's syndrome associated with COVID-19 has a low viral load. Viral load can be estimated using the ΔCt method (Ctₛₐₘₚₗₑ−Ct_{ref}). In some embodiments, a sample from a subject with a low viral load has a ΔCt>3. For example, a sample from a subject with a low viral load can have a ΔCt of about 3 to about 15. In some embodiments, the niclosamide compound, or a pharmaceutically acceptable salt thereof, is administered in combination with one or more additional therapeutic agents (e.g., any of the additional therapeutic agents described herein).

Also provided herein are methods for treating severe long hauler's syndrome associated with COVID-19 in a subject in need thereof, the method comprising administering an effective amount of a niclosamide compound, or a pharmaceutically acceptable salt thereof, to the subject. In some embodiments, a subject having severe COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, a subject having severe long hauler's syndrome associated with COVID-19 has one or more digestive symptoms. For example, the subject has one or more of diarrhea, abdominal pain, and vomiting. In some embodiments, a subject having severe long hauler's syndrome associated with COVID-19 has one or more of an elevated liver enzyme level, lower monocyte count, and longer prothrombin time. For example, an elevated liver enzyme level can include an AST or ALT level of >50 U/L. In some embodiments, a subject having severe long hauler's syndrome associated with COVID-19 has high levels of D-dimer. For example, a D-dimer level of at least about 2.0 μg/mL. See, e.g., Zhang et al. J Thromb Haemost. 2020; 10.1111/jth.14859. In some embodiments, a subject having severe COVID-19 has high levels of interleukin (IL)-6. For example, an IL-6 level of at least about 80 pg/mL. See, e.g., Zhang et al. J Thromb Haemost. 2020; 10.1111/jth.14859. See, e.g., Herold et al. medRxiv 2020.04.01.20047381; doi: doi.org/10.1101/2020.04.01.20047381. In some embodiments, a subject having severe long hauler's syndrome associated with COVID-19 has vitamin D deficiency. For example, the subject has a serum 25-hydroxyvitanim D (25(OH)D) level lower than about 30 nmol/L. In some embodiments, the subject has a serum 25(OH)D level of about 1 to about 30 nmol/L. In some embodiments, a subject having severe long hauler's syndrome associated with COVID-19 has a high viral load. In some embodiments, a sample from a subject with a high viral load has a ΔCt≤2. For example, a sample from a subject with a low viral load can have a ΔCt of about 2 to about −10. In some embodiments, the niclosamide compound, or a pharmaceutically acceptable salt thereof, is administered in combination with one or more additional therapeutic agents (e.g., any of the additional therapeutic agents described herein). Also provided herein are methods for treating mild long hauler's syndrome associated with COVID-19 in a subject in need thereof, the method comprising administering an effective amount of a niclosamide compound, or a pharmaceutically acceptable salt thereof, to the nasal cavity of the subject. In some embodiments, a subject having mild long hauler's syndrome associated with COVID-19 has: (i) a respiratory rate of ≤30 breaths per min; (ii) an oxygen saturation at rest of ≥93%; and (iii) a ratio of partial pressure of arterial oxygen to fractional concentration of oxygen inspired air of ≥300 mm Hg. In some embodiments, the subject does not have a severe disease complication. In some embodiments, the subject having mild long hauler's syndrome associated with COVID-19 has a low viral load (e.g., a ΔCt of about 3 to about 15). In some embodiments, the niclosamide compound, or a pharmaceutically acceptable salt thereof, is formulated as an intranasal spray, ointment, or gel (e.g., any of the intranasal compositions described herein).

Also provided herein are methods of treating a subject having long hauler's syndrome associated with COVID-19 that include: identifying a subject that has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and administering to the nasal cavity of the identified subject a treatment that includes a niclosamide compound, or a pharmaceutically acceptable salt thereof. In some embodiments, the subject does not have a severe disease complication. In some embodiments, the subject has a low viral load (e.g., a ΔCt of about 3 to about 15). In some embodiments, the niclosamide compound, or a pharmaceutically acceptable salt thereof, is formulated as an intranasal spray, ointment, or gel (e.g., any of the intranasal compositions described herein).

Also provided herein are methods for treating severe long hauler's syndrome associated with COVID-19 in a subject in need thereof, the method comprising administering an effective amount of a niclosamide compound, or a pharmaceutically acceptable salt thereof, to the lungs of the subject. In some embodiments, a subject having severe long hauler's syndrome associated with COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10). In some embodiments, the niclosamide compound, or a pharmaceutically acceptable salt thereof, is formulated for delivery by inhalation (e.g., any of the compositions for inhalation described herein).

Also provided herein are methods of treating a subject having long hauler's syndrome associated with COVID-19 that include: identifying a subject that has at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and administering to the lungs of the identified subject a treatment that includes a niclosamide compound, or a pharmaceutically acceptable salt thereof. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10). In some embodiments, the niclosamide compound, or a pharmaceutically acceptable salt thereof, is formulated for delivery by inhalation (e.g., any of the compositions for inhalation described herein).

Also provided herein are methods for treating severe long hauler's syndrome associated with COVID-19 in a subject in need thereof, the method comprising administering an effective amount of a niclosamide compound, or a pharmaceutically acceptable salt thereof, to the GI tract of the subject. In some embodiments, a subject having severe long hauler's syndrome associated with COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10). In some embodiments, the niclosamide compound, or a pharmaceutically acceptable salt thereof, is formulated for oral delivery.

Also provided herein are methods of treating a subject having long hauler's syndrome associated with COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a niclosamide compound, or a pharmaceutically acceptable salt thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a niclosamide compound, or a pharmaceutically acceptable salt thereof, to the lungs of the subject in which the subject has at least one of: (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication, e.g., a severe disease complication as described herein; or (e) administering additional doses of the niclosamide compound, or a pharmaceutically acceptable salt thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein).

Also provided herein are methods of treating a subject having long hauler's syndrome associated with COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a niclosamide compound, or a pharmaceutically acceptable salt thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a niclosamide compound, or a pharmaceutically acceptable salt thereof, to the GI tract of the subject in which the subject has at least one of: (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication, e.g., a severe disease complication as described herein; or (e) administering additional doses of the niclosamide compound, or a pharmaceutically acceptable salt thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein).

Also provided herein are methods of treating a subject having long hauler's syndrome associated with COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a niclosamide compound, or a pharmaceutically acceptable salt thereof, to the nasal cavity of the subject; (c) after (a) and (b), identifying whether the subject has at least one of: (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication; and (d) administering one or more doses of a niclosamide compound, or a pharmaceutically acceptable salt thereof, to the GI tract of the subject and administering one or more doses of a niclosamide compound, or a pharmaceutically acceptable salt thereof, to the lungs of the subject in which the subject has at least one of: (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication, e.g., a severe disease complication as described herein; or (e) administering additional doses of the niclosamide compound, or a pharmaceutically acceptable salt thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of ≥300 mm Hg. In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein).

Also provided herein are methods of treating a subject having long hauler's syndrome associated with COVID-19 that include: (a) identifying a subject having at least one of (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication; and (b) administering one or more doses of a niclosamide compound, or a pharmaceutically acceptable salt thereof, to the lungs of the subject; (c) after (a) and (b), identifying whether the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (d) administering one or more doses of a niclosamide compound, or a pharmaceutically acceptable salt thereof, to the nasal cavity of the subject in which the subject has (i) a respiratory rate of <30 breaths per min; (ii) an oxygen saturation at rest of ≥93%; and (iii) a ratio of partial pressure of arterial oxygen to fractional concentration of oxygen inspired air of ≥300 mm Hg; or (e) administering additional doses of the niclosamide compound, or a pharmaceutically acceptable salt thereof, to the lungs of the subject in which the subject has at least one of (i) respiratory distress (i.e., ≥30 breaths per min); (ii) an oxygen saturation at rest of <93%; (iii) a ratio of partial pressure of arterial oxygen to fractional concentration of oxygen inspired air of <300 mm Hg; and (iv) a severe disease complication, e.g., a severe disease complication as described herein. In some embodiments, the subject identified in step (c) does not have a severe disease complication. In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein). Also provided herein are methods of treating a subject having long hauler's syndrome associated with COVID-19 that include: (a) identifying a subject having a low viral load (e.g., a ΔCt of about 3 to about 15); and (b) administering one or more doses of a niclosamide compound, or a pharmaceutically acceptable salt thereof, to the nasal cavity of the subject; (c) after (a) and (b), identifying whether the subject has a high viral load (e.g., a ΔCt of about 2 to about −10); and (d) administering one or more doses of a niclosamide compound, or a pharmaceutically acceptable salt thereof, to the lungs of the subject in which the subject has a high viral load (e.g., a ΔCt of about 2 to about −10); or (e) administering additional doses of the niclosamide compound, or a pharmaceutically acceptable salt thereof, to the nasal cavity of the subject in which the subject has a low viral load (e.g., a ΔCt of about 3 to about 15). In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein).

Also provided herein are methods of treating a subject having long hauler's syndrome associated with COVID-19 that include: (a) identifying a subject having a low viral load (e.g., a ΔCt of about 3 to about 15); and (b) administering one or more doses of a niclosamide compound, or a pharmaceutically acceptable salt thereof, to the nasal cavity of the subject; (c) after (a) and (b), identifying whether the subject has a high viral load (e.g., a ΔCt of about 2 to about −10); and (d) administering one or more doses of a niclosamide compound, or a pharmaceutically acceptable salt thereof, to the GI tract of the subject in which the subject has a high viral load (e.g., a ΔCt of about 2 to about −10); or (e) administering additional doses of the niclosamide compound, or a pharmaceutically acceptable salt thereof, to the nasal cavity of the subject in which the subject has a low viral load (e.g., a ΔCt of about 3 to about 15). In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein).

Also provided herein are methods of treating a subject having long hauler's syndrome associated with COVID-19 that include: (a) identifying a subject having a low viral load (e.g., a ΔCt of about 3 to about 15); and (b) administering one or more doses of a niclosamide compound, or a pharmaceutically acceptable salt thereof, to the nasal cavity of the subject; (c) after (a) and (b), identifying whether the subject has a high viral load (e.g., a ΔCt of about 2 to about −10); and (d) administering one or more doses of a niclosamide compound, or a pharmaceutically acceptable salt thereof, to the GI tract of the subject and administering one or more doses of a niclosamide compound, or a pharmaceutically acceptable salt thereof, to the lungs of the subject in which the subject has a high viral load (e.g., a ΔCt of about 2 to about −10); or (e) administering additional doses of the niclosamide compound, or a pharmaceutically acceptable salt thereof, to the nasal cavity of the subject in which the subject has a low viral load (e.g., a ΔCt of about 3 to about 15). In some embodiments, the subject identified in step (a) does not have a severe disease complication. In some embodiments, wherein the niclosamide compound, or a pharmaceutically acceptable salt thereof is administered to the nasal cavity of the subject, the niclosamide compound, or the pharmaceutically acceptable salt thereof, is formulated as an intranasal spray, ointment, or gel (e.g., any of the intranasal compositions described herein). In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein).

Also provided herein are methods of treating a subject having long hauler's syndrome associated with COVID-19 that include: (a) identifying a subject having long hauler's syndrome associated with COVID-19 has a high viral load (e.g., a ΔCt of about 2 to about −10); and (b) administering one or more doses of a niclosamide compound, or a pharmaceutically acceptable salt thereof, to the lungs of the subject; (c) after (a) and (b), identifying whether the subject has a low viral load (e.g., a ΔCt of about 3 to about 15); and (d) administering one or more doses of a niclosamide compound, or a pharmaceutically acceptable salt thereof, to the nasal cavity of the subject in which the subject has a low viral load (e.g., a ΔCt of about 3 to about 15); or (e) administering additional doses of the niclosamide compound, or a pharmaceutically acceptable salt thereof, to the lungs of the subject in which the subject has a high viral load (e.g., a ΔCt of about 2 to about −10). In some embodiments, the subject identified in step (c) does not have a severe disease complication. In some embodiments, wherein the niclosamide compound, or a pharmaceutically acceptable salt thereof is administered to the GI tract of the subject, the niclosamide compound, or the pharmaceutically acceptable salt thereof, is formulated as an oral composition (e.g., any of the oral compositions described herein). In some embodiments, wherein the niclosamide compound, or a pharmaceutically acceptable salt thereof is administered to the lungs of the subject, the niclosamide compound, or the pharmaceutically acceptable salt thereof, is formulated for delivery by inhalation (e.g., any of the compositions for inhalation described herein).

Also provided herein are methods of treating a subject having long hauler's syndrome associated with COVID-19 that include: identifying a subject that has a D-dimer level of at least about 2.0 μg/mL (e.g., a subject that has been identified or diagnosed as having a D-dimer level of at least about 2.0 μg/mL through the use of a regulatory agency-approved, e.g., FDA-approved, kit for detecting D-dimer levels in a subject or a sample from the subject); and administering to the identified subject a treatment that includes a niclosamide compound, or a pharmaceutically acceptable salt thereof. In some embodiments, the subject has thrombosis. In some embodiments, the niclosamide compound, or a pharmaceutically acceptable salt thereof, is formulated for delivery to endothelial cells.

In some embodiments, provided herein are methods of treating a subject having long hauler's syndrome associated with COVID-19 that include: identifying a subject that has a D-dimer level of at least about 2.0 μg/mL (e.g., a subject that has been identified or diagnosed as having a D-dimer level of at least about 2.0 μg/mL through the use of a regulatory agency-approved, e.g., FDA-approved, kit for detecting D-dimer levels in a subject or a sample from the subject); and administering to the identified subject a treatment that includes a niclosamide compound, or a pharmaceutically acceptable salt thereof and an anticoagulation agent (e.g., any of the anticoagulation agent described herein). In some embodiments, the subject has thrombosis. In some embodiments, the niclosamide compound, or a pharmaceutically acceptable salt thereof, is formulated for delivery to endothelial cells.

In some embodiments, the method includes obtaining a sample from the subject. In some embodiments, the sample is a blood sample.

Also provided herein are methods of treating a subject having long hauler's syndrome associated with COVID-19 that include: identifying a subject that has an IL-6 level of at least about 80 pg/mL (e.g., a subject that has been identified or diagnosed as having an IL-6 level of at least about 80 pg/mL through the use of a regulatory agency-approved, e.g., FDA-approved, kit for detecting IL-6 levels in a subject or a sample from the subject); and administering to the identified subject a treatment that includes a niclosamide compound, or a pharmaceutically acceptable salt thereof. In some embodiments, the subject has cytokine storm syndrome.

In some embodiments, provided herein are methods of treating a subject having long hauler's syndrome associated with COVID-19 that include: identifying a subject that has an IL-6 level of at least about 80 pg/mL (e.g., a subject that has been identified or diagnosed as having an IL-6 level of at least about 80 pg/mL through the use of a regulatory agency-approved, e.g., FDA-approved, kit for detecting IL-6 levels in a subject or a sample from the subject); and administering to the lungs of the identified subject a treatment that includes a niclosamide compound, or a pharmaceutically acceptable salt thereof, and an IL-6 targeted therapy (e.g., any of the IL-6 targeted therapies described herein). In some embodiments, the subject has cytokine storm syndrome.

In some embodiments, the method includes obtaining a sample from the subject. In some embodiments, the sample is a blood sample.

Also provided herein are methods of treating a subject having long hauler's syndrome associated with COVID-19 that include: identifying a subject that has a serum 25(OH)D level of less than about 30 nmol/L (e.g., a subject that has been identified or diagnosed as having a serum 25(OH)D level of less than about 30 nmol/L through the use of a regulatory agency-approved, e.g., FDA-approved, kit for detecting 25(OH)D levels in a subject or a sample from the subject); and administering to the identified subject a treatment that includes a niclosamide compound, or a pharmaceutically acceptable salt thereof.

In some embodiments, provided herein are methods of treating a subject having long hauler's syndrome associated with COVID-19 that include: identifying a subject that has a serum 25(OH)D level of less than about 30 nmol/L (e.g., a subject that has been identified or diagnosed as having a serum 25(OH)D level of less than about 30 nmol/L through the use of a regulatory agency-approved, e.g., FDA-approved, kit for detecting 25(OH)D levels in a subject or a sample from the subject); and administering to the identified subject a treatment that includes a niclosamide compound, or a pharmaceutically acceptable salt thereof and vitamin D.

In some embodiments, the method includes obtaining a sample from the subject. In some embodiments, the sample is a blood sample.

In some embodiments, the patient is identified or diagnosed as having long haulers syndrome associated with Covid-19 through machine learning, e.g., by an immune based prediction. In certain embodiments, a bioinformatics approach is used based on immune subset profiling and a 14-plex cytokine panel that is run on patients. One score for the long hauler's syndrome associated with Covid-19 is defined as S1=(IFN-γ+IL-2)/CCL4-MIP-1β. Another score is defined as S2=(10*IL-10+IL-6)−(IL-2+IL-8). Patients with a severe case present excessive inflammation and dysregulated T cell activation, recruitment, and counteracting activities.

Combination Therapy

In some embodiments, the methods and compositions described herein are suitable for use in combination therapy with various other therapeutic regimens. In certain embodiments, the niclosamide compounds, or pharmaceutically acceptable salts thereof, and methods described herein can be used to treat side effects produced by such therapeutic regimens.

In some embodiments, the methods and compositions described herein are suitable for use in combination therapy with one or more additional therapeutic agents.

In certain embodiments, the one or more additional therapeutic agents is administered to the subject prior to contacting with or administering the niclosamide compound, or a pharmaceutically acceptable salt thereof, (e.g., about one hour prior, or about 6 hours prior, or about 12 hours prior, or about 24 hours prior, or about 48 hours prior, or about 1 week prior, or about 1 month prior).

In other embodiments, the one or more additional therapeutic agents is administered to the subject at about the same time as contacting with or administering the niclosamide compound, or a pharmaceutically acceptable salt thereof. By way of example, the second therapeutic agent or regimen and the niclosamide compound, or a pharmaceutically acceptable salt thereof, are provided to the subject simultaneously in the same dosage form. As another example, the second therapeutic agent or regimen and the niclosamide compound, or a pharmaceutically acceptable salt thereof, are provided to the subject concurrently in separate dosage forms.

In still other embodiments, the one or more additional therapeutic agents is administered to the subject after contacting with or administering the niclosamide compound, or a pharmaceutically acceptable salt thereof, (e.g., about one hour after, or about 6 hours after, or about 12 hours after, or about 24 hours after, or about 48 hours after, or about 1 week after, or about 1 month after).

In some embodiments, one or more therapies can be used in combination with the materials and/or methods described herein. In some embodiments, a combination therapy can include one or more of a macrolide antibiotic, an anti-malarial agent, an anti-diabetic agent, an angiotensin receptor inhibitor, an angiotensin-converting enzyme (ACE) inhibitor, a statin, a polymerase inhibitor (e.g., a RNA-dependent RNA polymerase inhibitor), a protease inhibitor, a neuraminidase inhibitor, a fusion inhibitor, a transmembrane protease serine 2 (TMPRSS2) inhibitor, a broad-spectrum antiviral agent, a JAK-STAT pathway inhibitor, a DNA synthesis inhibitor, a phosphodiesterase 5 (PDE5) inhibitor, a monoclonal antibody, passive antibody therapy, recombinant human angiotensin-converting enzyme 2 (rhACE2), traditional Chinese medicine, an anticoagulation agent, a pharmaceutically acceptable salt or solvate of any thereof, or two or more of any thereof. In some embodiments, the combination therapy is an IL-6 targeted therapy. Non-limiting examples, of IL-6 targeted therapies include tocilizumab and siltuximab. In some embodiments, the combination therapy is an anticoagulation agent. Non-limiting examples, of an anticoagulation agents include warfarin, heparin, rivaroxaban, dabigatran, apixaban, edoxaban, enoxaparin, and fondaparinux. In some embodiments, the macrolide antibiotic and/or anti-malarial agent is a lysosomotropic agent. Non-limiting examples of lysosomotropic agents include azithromycin, hydroxychloroquine, chloroquine, and ammonium chloride. Non-limiting examples of an anti-diabetic agent include a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor. In some embodiments, the biguanide is metformin. A non-limiting example of an angiotensin receptor inhibitor includes a sartan (e.g., eprosartan, olmesartan, olmesartan medoxomil, valsartan, candesartan, candesartan cilexetil, losartan, telmisartan, irbesartan, BRA-657, and azilsartan medoxomil). Non-limiting examples of an ACE inhibitor include: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. Non-limiting examples of a statin include atorvastatin, fluvastatin, lovastatin, pitavastatin, pravastatin, rosuvastatin, and simvastatin, cerivastatin, and mevastatin. Non-limiting examples of polymerase inhibitors include ganciclovir, valganciclovir, and RNA-dependent RNA polymerase (RdRP) inhibitors (e.g., remdesivir, ribavirin, and favipiravir). Non-limiting examples of protease inhibitors include lopinavir, ritonavir, indinavir, atazanavir, nelfinavir, darunavir, tipranavir, amprenavir, and fosamprenavir. A non-limiting example of a neuraminidase inhibitor is oseltamivir. A non-limiting example of a fusion inhibitor is umifenovir. A non-limiting example of a TMPRSS2 inhibitor is camostat. Non-limiting examples of broad-spectrum antiviral agents include nitazoxanide, chloroquine, hydroxychloroquine, and interferon (e.g., interferon alfa). Non-limiting examples of JAK-STAT pathway inhibitors include baricitinib, fedratinib, and ruxolitinib. Non-limiting examples of DNA synthesis inhibitors include tenofovir disoproxil and lamivudine. A non-limiting example of a PDE5 inhibitor is sildenafil. A non-limiting example of traditional Chinese medicine is huaier extract. See, e.g., Liu, Cynthia, et al. “Research and Development on Therapeutic Agents and Vaccines for COVID-19 and Related Human Coronavirus Diseases.” (2020). doi/10.1021/acscentsci.0c00272; Lai, Chih-Cheng, et al. “Severe acute respiratory syndrome coronavirus 2 (SARS-CoV-2) and coronavirus disease-2019 (COVID-19): the epidemic and the challenges.” International journal of antimicrobial agents (2020): 105924; Chang, Yu-Chuan, et al. “Potential therapeutic agents for COVID-19 based on the analysis of protease and RNA polymerase docking.” (2020). doi: 10.20944/preprint5202002.0242.v1; NCT04252885; NCT04306497; NCT04287686; NCT04307693; NCT04292899; NCT04304313; NCT04291053; Ko W C, Rolain J M, Lee N Y, et al. Arguments in favor of remdesivir for treating SARS-CoV-2 infections [published online ahead of print, 2020 Mar. 5]. Int J Antimicrob Agents. 2020; 105933. doi:10.1016/j.ijantimicag.2020.105933; Dhama, K., et al. (2020). Coronavirus Disease 2019—COVID-19. doi: 10.20944/preprints202003.0001.v1; Stebbing, Justin, et al. “COVID-19: combining antiviral and anti-inflammatory treatments.” The Lancet Infectious Diseases (2020); Yang, Naidi, and Han-Ming Shen. “Targeting the Endocytic Pathway and Autophagy Process as a Novel Therapeutic Strategy in COVID-19.” Int J Biol Sci 16.10 (2020): 1724-1731; Casadevall, Arturo, and Liise-anne Pirofski. “The convalescent sera option for containing COVID-19.” The Journal of Clinical Investigation 130.4 (2020); Shanmugaraj, Balamurugan, et al. “Perspectives on monoclonal antibody therapy as potential therapeutic intervention for Coronavirus disease-19 (COVID-19).” Asian Pacific journal of allergy and immunology (2020); and Xu, Jimin, et al. “Broad Spectrum Antiviral Agent Niclosamide and Its Therapeutic Potential.” ACS Infectious Diseases (2020), each of which is incorporated by reference herein in its entirety.

Provided herein are methods for treating long hauler's syndrome associated with COVID-19 in a subject in need thereof, the method comprising administering a niclosamide compound, or a pharmaceutically acceptable salt thereof, and a lysosomotropic agent to the subject. In some embodiments, the lysosomotropic agent is selected from azithromycin, hydroxychloroquine, chloroquine, ammonium chloride, and a combination thereof.

In some embodiments, methods provided herein for treating long hauler's syndrome associated with COVID-19 in a subject in need thereof include administering a niclosamide compound, or a pharmaceutically acceptable salt thereof, and azithromycin. In some embodiments, about 500 mg azithromycin is administered to the subject once per day. In some embodiments, about 250 mg azithromycin is administered to the subject once per day. In some embodiments, about 500 mg azithromycin is administered to the subject on Day 1 and about 250 mg azithromycin is administered to the subject once per day on Days 2-5. See, e.g., Gautret et al. Int J Antimicrob Agents. 2020 Mar. 20:105949.

In some embodiments, methods provided herein for treating long hauler's syndrome associated with COVID-19 in a subject in need thereof include administering a niclosamide compound, or a pharmaceutically acceptable salt thereof, and hydroxychloroquine. In some embodiments, about 200 mg hydroxychloroquine is administered to the subject three times per day. In some embodiments, about 200 mg hydroxychloroquine is administered to the subject three times per day for about 10 days. See, e.g., Gautret et al. Int J Antimicrob Agents. 2020 Mar. 20:105949.

In some embodiments, methods provided herein for treating long hauler's syndrome associated with COVID-19 in a subject in need thereof include administering a niclosamide compound, or a pharmaceutically acceptable salt thereof, and chloroquine.

In some embodiments, methods provided herein for treating long hauler's syndrome associated with COVID-19 in a subject in need thereof include administering a niclosamide compound, or a pharmaceutically acceptable salt thereof, azithromycin, and hydroxychloroquine.

In some embodiments, methods provided herein for treating long hauler's syndrome associated with COVID-19 in a subject in need thereof include administering a niclosamide compound, or a pharmaceutically acceptable salt thereof, azithromycin, and chloroquine.

In some embodiments, the chloroquine is chloroquine phosphate (e.g., ARALEN®). In some embodiments, the hydroxychloroquine is hydroxychloroquine sulfate (e.g., PLAQUENIL®).

Also provided herein are methods for treating long hauler's syndrome associated with COVID-19 in a subject in need thereof, the methods comprising administering a niclosamide compound, or a pharmaceutically acceptable salt thereof, and an anti-diabetic agent. In some embodiments, the anti-diabetic agent is metformin.

Also provided herein are methods for treating long hauler's syndrome associated with COVID-19 in a subject in need thereof, the methods comprising administering a niclosamide compound, or a pharmaceutically acceptable salt thereof, and an angiotensin receptor inhibitor. In some embodiments, the angiotensin receptor inhibitor is selected from eprosartan, olmesartan, valsartan, candesartan, losartan, telmisartan, irbesartan, azilsartan medoxomil, and a combination thereof.

Also provided herein are methods for treating long hauler's syndrome associated with COVID-19 in a subject in need thereof, the methods comprising administering a niclosamide compound, or a pharmaceutically acceptable salt thereof, and a statin. In some embodiments, the statin is selected from atorvastatin, fluvastatin, lovastatin, pitavastatin, pravastatin, rosuvastatin, and simvastatin, cerivastatin, mevastatin, and a combination thereof.

Also provided herein are methods for treating long hauler's syndrome associated with COVID-19 in a subject in need thereof, the method comprising administering a niclosamide compound, or a pharmaceutically acceptable salt thereof, and an IL-6 targeted therapy to the subject. In some embodiments, the subject has cytokine storm syndrome. In some embodiments, the IL-6 targeted agent is selected from tocilizumab, siltuximab, and a combination thereof.

Also provided herein are methods for treating long hauler's syndrome associated with COVID-19 in a subject in need thereof, the method comprising administering a niclosamide compound, or a pharmaceutically acceptable salt thereof, and vitamin D to the subject. In some embodiments, the subject has severe or mild long hauler's syndrome associated with COVID-19.

Niclosamide Compounds

Chemical Purity

In some embodiments, the niclosamide compounds (e.g., niclosamide) have a chemical purity of greater than about 95%, greater than about 96%, greater than about 97%, greater than about 98% or greater than about 99.0%; e.g., greater than about 99.5%; or greater than about 99.7%; or greater than about 99.8%.

In some embodiments, the niclosamide compounds (e.g., niclosamide) have less than about 75 ppm, less than about 60 ppm, or less than about 45 ppm of 5-chloro-salicylic acid; e.g., less than about 30 ppm of 5-chloro-salicylic acid.

In some embodiments, the compound has less than about 125 ppm, less than about 100 ppm, less than about 75 ppm, or less than about 50 ppm of 2-chloro-4 nitro-aniline. In certain embodiments, the compound has less than about 10 ppm of 2-chloro-4 nitro-aniline.

In some embodiments, the compound has less than about 45 ppm of 5-chloro-salicylic acid and less than about 50 ppm of 2-chloro-4 nitro-aniline.

In some embodiments, the compound has less than about 30 ppm of 5-chloro-salicylic acid and less than about 10 ppm of 2-chloro-4 nitro-aniline.

In some embodiments, the compound has less than about 1%, less than about 0.5% or less than about 0.05% water. In certain embodiments, the compound is substantially free of hydrated niclosamide solid forms. As a non-limiting example, the compound can be anhydrous niclosamide.

In some embodiments, purification can be carried out according to the following process. Acetone and crude niclosamide are mixed in a vessel and heated to reflux (˜56° C.) until solids dissolve. The solution is clarified by filtration and transferred to a second vessel, heated to 45° C. to 55° C. to dissolve the solids, cooled to −5° C. to 5° C. and stirred at this temperature for at least 2 hours. The solids are filtered and washed with acetone. Crystallized niclosamide is obtained after vacuum drying of the solids at 70° C. IPC LOD testing is performed on the dry solids with a specification of <1.0%. If the LOD results are >1.0% the drying step may be repeated two additional times. IPC testing is also performed to ensure the level of the starting material 2-chloro-4-nitroaniline is <100 ppm. If the level of 2-chloro-4-nitroaniline is >100 ppm, a second crystallization may be performed.

In some embodiments, purity analysis can be achieved according to the following procedure. Chromatograph: UPLC system consisting of pump, diode array; detector, autosampler, auto injector, and column cooler/heater, or equivalent. Column: Agilent Poroshell 120 EC-C18 column, 4.6×50 mm, 2.7 μm or equivalent. Column Temperature: 35° C. Mobile phase A: 20 mM ammonium acetate (pH 5.50). Mobile phase B: MeOH:ACN (70:30, v/v). Diluent: MeOH:DMSO (70:30, v/v).

| Time | Solvent A | Solvent B |
|---|---|---|
| (min.) | (%) | (%) |
| 0.0 | 75 | 25 |
| 1.0 | 75 | 25 |
| 21.0 | 30 | 70 |

Flow rate: 1.0 ml/min. Injected volume: 3.00 μl. Preparation of standard and sample solutions. Niclosamide Standard Solutions: Concentration of this solution is nominally 0.8 mg/mL. Retention times: 5-Chlorosalicylic acid (2.9 minutes); 2-Chloro-4-nitroaniline (7.0 minutes); and Niclosamide (18.8 minutes).

Particle Size

In some embodiments, the compound has a reduced particle size (e.g., as achieved by techniques including but not limited to milling).

In some embodiments, niclosamide compounds having reduced particle size can be prepared by jet milling, e.g., using CMTI equipment NGMP-Mill-A, a 2-inch, pancake micronizer manufactured by Sturtevant; a flexible containment unit was used during the milling process (Mill and Venturi pressure both=50 psi; feed rate 96.0 g/hour).

In some embodiments of the foregoing, the compound has a particle size range of from about 0.1 μm to about 30 μm. In certain embodiments, the compound has a particle size range of from about 0.1 μm to about 20 μm. In certain embodiments, the compound has a particle size range of from about 0.1 μm to about 10 μm.

The term “particle size distribution” of a powder, or granular material, or particles dispersed in fluid, as used within this application, is a list of values or a mathematical function that defines the relative amounts of particles present, sorted according to size. The d(0.1), d(0.5) and d(0.9) values indicate that 10%, 50% and 90% of the particles measured were less than or equal to the size stated. For example, values of d(0.1)=0.6, d(0.5)=3.1 and d(0.9)=7.3 mean that 10% of the particles were less than or equal to 0.6 μm, 50% were less than or equal to 3.1 μm, and 90% were less than or equal to 7.3 μm.

Particle Size Distribution (PSD) can be determined by laser diffraction technique, e.g., using a “MALVERN MASTERSIZER 2000” (standard range between 0.020 and 2000.0 microns), model “APA 2000”, equipped with “Hydro 2000 sm” as dispersing unit. A representative procedure includes: approximately 50 mg of Niclosamide is dispersed manually into 25 ml of water; after dispersion the sample was sonicated with external ultrasound for two minutes (Ultrasonic frequency; 37 kHz—Elmasonic S100 (H)—Elma Schmidbauer GmbH, Germany); the following operative conditions/machine parameters are taken into account: Dispersant: Water+3 drops of Tyloxapol 1.5%; Background measurement time: 10 seconds; Number of measurements cycles: 3 (to obtain average value); Stir speed (dispersing unit): 1500 rpm.

In some embodiments, the compound has a particle size distribution D(0.9) of from about 1.0 μm to about 15.0 μm. In certain embodiments, the compound has a particle size distribution D(0.9) of from about 1.0 μm to about 10.0 μm. In certain embodiments, the compound has a particle size distribution D(0.9) of from about 6.0 μm to about 8.0 μm (e.g., about 7.3 μm (e.g., 7.3 μm)). In other embodiments, the compound has a particle size distribution D(0.9) of from about 2.2 μm to about 3.2 μm.

In some embodiments, the compound has a particle size distribution D(0.1) of from about 0.1 μm to about 1.5 μm. In certain embodiments, the compound has a particle size distribution D(0.1) of from about 0.1 μm to about 1.0 μm. In certain embodiments, the compound has a particle size distribution D(0.1) of from about 0.3 μm to about 0.9 μm. In certain embodiments, the compound has a particle size distribution D(0.1) of from about 0.45 to about 0.75 μm (e.g., about 0.6 μm (e.g., 0.6 μm)).

In some embodiments, the compound has a particle size distribution D(0.5) of from about 0.5 μm to about 6.0 μm. In certain embodiments, the compound has a particle size distribution D(0.5) of from about 1.0 μm to about 4.0 μm. In certain embodiments, the compound has a particle size distribution D(0.5) of from about 1.0 μm to about 2.0 μm. In certain other embodiments, the compound has a particle size distribution D(0.5) of from about 2.5 μm to about 3.5 μm (e.g., about 3.1 μm (e.g., 3.1 μm)).

The parameter D(0.1) as used herein refers to the mesh size of a single notional sieve allowing 10% of the total of all particles of the sample to pass. Thus D(0.1)=0.1-1.5 means that the upper limit of the particle size range defining the 10% of smallest particles in the sample is between 0.1 μm to 1.5 μm. Thus 10% of the total particles have a particle size of not more than D(0.1) meaning in this case that they have a maximum size of 0.1 μm to 1.5 μm.

The parameter D(0.5) refers to the mesh size of a single notional sieve allowing 50% of the total of all particles of the sample to pass. Thus D(0.5)=0.5-6.0 μm means that the upper limit of the particle size range defining the notional half of the sample containing the smaller particles is between 0.5 μm to 6.0 μm. Thus, 50% of the total of all particles have a particle size of not more than D(0.5) meaning in this case that they have a maximum size of 0.5 to 6.0 μm.

The parameter D(0.9) refers to the mesh size of a single notional sieve allowing 90% of the total of all particles of the sample to pass i.e. only 10% of the sample is retained. Thus, D(0.9)=1.0-15.0 μm means that the lower limit of the particle size range defining the 10% of largest particles in the sample is between 1.0 μm to 15.0 μm. Thus 90% of all particles have a particle size of not more than D(0.9) meaning in this case that they have a maximum size of 1.0 to 15.0 μm.

In some embodiments, the compound has less than or equal to any of about 5%, about 4%, about 3%, about 2%, about 1%, about 0.5% or about 0.05% water (e.g., as determined by Karl Fisher technique). In certain embodiments, the compound is substantially free of hydrated niclosamide solid forms. As a non-limiting example, the compound can be anhydrous niclosamide.

In some embodiments, the compound is crystalline.

In some embodiments, the compound has a specific surface area of from about 5 m²/g to about 10 m²/g.

Non-Limiting Combination

### Non-Limiting Combinations [A]

In some embodiments, the compound has a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound has a particle size distribution D(0.9) of from about 7.0 μm to about 7.5 μm (e.g., about 7.3 μm), a particle size distribution D(0.5) of from about 2.5 μm to about 4.0 μm (e.g., about 3.1 μm), and a particle size distribution D(0.1) of from about 0.45 μm to about 0.75 μm (e.g., about 0.6 μm).

In some embodiments, the compound has a particle size distribution D(0.9) of about 7.3 μm, a particle size distribution D(0.5) of about 3.1 μm, and a particle size distribution D(0.1) of about 0.6 μm.

In some embodiments, the compound has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 95%, greater than about 96%, greater than about 97%, greater than about 98% or greater than about 99.0%. (e.g., greater than about 99.5%; or greater than about 99.7%; or greater than about 99.8%); a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a chemical purity of greater than about 99.0%, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size range of from about 0.1 μm to about 30 μm, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a chemical purity of greater than about 99.0%, a particle size range of from about 0.1 μm to about 30 μm, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size range of from about 0.1 μm to about 30 μm, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In certain embodiments of [A], the compound has a particle size distribution D(0.5) of from about 2.5 μm to about 3.5 μm.

In certain embodiments of [A], the compound has a particle size distribution D(0.5) of from about 1.0 μm to about 2.0 μm.

In certain embodiments of [A], the compound has a chemical purity of greater than about 95%, greater than about 96%, greater than about 97%, greater than about 98% or greater than about 99.0%. (e.g., greater than about 99.5%; or greater than about 99.7%; or greater than about 99.8%).

In certain embodiments of [A], the compound has less than about 45 ppm of 5-chloro-salicylic acid; or less than about 30 ppm of 5-chloro-salicylic acid.

In certain embodiments of [A], the compound has less than about 50 ppm of 2-chloro-4 nitro-aniline; or less than about 10 ppm of 2-chloro-4 nitro-aniline.

In certain embodiments of [A], the compound has less than about 45 ppm of 5-chloro-salicylic acid and less than about 50 ppm of 2-chloro-4 nitro-aniline; or less than about 30 ppm of 5-chloro-salicylic acid and less than about 10 ppm of 2-chloro-4 nitro-aniline.

In certain embodiments of [A], the compound has less than about 0.05% water.

In certain embodiments of [A], the compound is substantially free of hydrated niclosamide solid forms.

In certain embodiments of [A], the compound is anhydrous niclosamide.

In certain embodiments of [A], the compound is crystalline.

In certain embodiments of [A], the compound has a specific surface area of from about 5 m²/g to about 10 m²/g.

Cocrystals of Niclosamide Compounds

Overview

In some embodiments, the niclosamide compounds (e.g., niclosamide) can be in the form of a cocrystal that includes (i) a niclosamide compound (e.g., niclosamide) or a pharmaceutically acceptable salt thereof; and (ii) one or more pharmaceutically acceptable coformers. The term “co-crystal” as used herein refers to a crystalline material comprised of two or more unique solids at room temperature in a stoichiometric or non-stoichiometric ratio, which are held together in the crystal lattice by one or more non-covalent interactions (e.g., hydrogen bonds, pi-stacking, guest-host complexation and van der Waals interactions).

In some embodiments, at least one of the one or more non-covalent interactions is a hydrogen bond. In certain of these embodiments, the chemical entity is the hydrogen bond donor, and one of one or more coformers is the hydrogen bond acceptor. In other embodiments, the chemical entity is the hydrogen bond acceptor, and one of one or more coformers is the hydrogen bond donor.

The co-crystals described herein can include one or more solvate (e.g., water or an organic solvent containing one or more hydroxyl groups, e.g., a C₁-C₆ alcohol or diol, e.g., a C₁-C₆ alcohol or diol, e.g., ethanol or propylene glycol) molecules in the crystalline lattice. However, solvates of chemical entities that do not further comprise a coformer (e.g., a solid conformer) are not encompassed by the co-crystal definition set forth in this disclosure.

In some embodiments, the cocrystal includes more than one coformer. For example, two, three, four, five, or more co formers can be incorporated in a co-crystal with the chemical entity. The ratio of the chemical entity to each of the one or more pharmaceutically acceptable coformers may be stoichiometric or non-stoichiometric. As a non-limiting example, 1:1, 1:1.5 and 1:2 ratios of chemical entity:coformer are contemplated.

The niclosamide compounds (e.g., niclosamide) and each of the one or more pharmaceutically acceptable coformers may each be independently specified as a free form, or more specifically, a free acid, free base, or zwitter ion; a salt, or more specifically for example, an inorganic base addition salt such as sodium, potassium, lithium, calcium, magnesium, ammonium, aluminum salts or organic base addition salts, or an inorganic acid addition salts such as HBr, HCl, sulfuric, nitric, or phosphoric acid addition salts or an organic acid addition salt such as acetic, proprionic, pyruvic, malanic, succinic, malic, maleic, fumaric, tartaric, citric, benzoic, methanesulfonic, ethanesulforic, stearic or lactic acid addition salt; an anhydrate or hydrate of a free form or salt, or more specifically, for example, a hemihydrate, monohydrate, dihydrate, trihydrate, quadrahydrate, pentahydrate; or a solvate of a free form or salt.

Coformers

In some embodiments, at least one of the one or more pharmaceutically acceptable coformers can form one or more hydrogen bonds with the chemical entity in the cocrystal. In some embodiments, at least one of the one or more pharmaceutically acceptable coformers can accept one or more hydrogen bonds from the chemical entity in the cocrystal. In some embodiments, at least one of the one or more pharmaceutically acceptable coformers can form one or more hydrogen bonds with the chemical entity in the cocrystal, and at least one of the one or more pharmaceutically acceptable coformers can accept one or more hydrogen bonds from the chemical entity in the cocrystal.

In some embodiments, at least one of the one or more pharmaceutically acceptable coformers comprises one or more functional groups selected from the group consisting of: ether, thioether, hydroxy, sulfhydryl, aldehyde, ketone, thioketone, nitrate ester, phosphate ester, thiophosphate ester, ester, thioester, sulfate ester, carboxylic acid, phosphonic acid, phosphinic acid, sulfonic acid, amido, primary amine, secondary amine, ammonia, tertiary amino, sp2 amino, thiocyanate, cyanamide, oxime, nitrile, diazo, haloalkyl, nitro, heterocyclic ring, heteroaryl ring, epoxide, peroxide, and hydroxamic acid.

In some of these embodiments, at least one of the one or more pharmaceutically acceptable coformers can be a compound having any one of formulas (I), (XVIII)-(XXV), and XXVII, (e.g., formula XXIV or XXV) as described in U.S. Pat. No. 10,292,951 which is incorporated herein by reference in its entirety; or any one of the compounds delineated above. In certain of these embodiments, at least one of the one or more pharmaceutically acceptable coformers can be a niclosamide analogue having any one of formulas (I), (XVIII)-(XXV), and XXVII (e.g., formula XXIV or XXV; or XXVI) as described in U.S. Pat. No. 10,292,951 which is incorporated herein by reference in its entirety; or any one of the compounds specifically delineated above.

In some embodiments, the coformer can be any one or more additional therapeutic agents as described herein.

In some embodiments, the co-former is selected from the group consisting of: a sphingosine 1-phosphate (S1P) receptor modulator; a steroidal anti-inflammatory agent; a non-steroidal anti-inflammatory agent; a receptor-interacting protein kinase 1 (RIPK1) inhibitor; an EP4 modulator; a toll-like receptor (e.g., TLR4, TLR9) modulator; a Janus kinase (JAK) inhibitor; a lanthionine synthetase C-like 2 (LANCL2) modulator; a phosphatidylcholine; an integrin (e.g., α4 Integrin) modulator; a Smad7 modulator; a phosphodiesterase 4 (PDE4) modulator; a tumor progression locus 2 (TPL2) inhibitor; a tyrosine kinase 2 (TYK2) inhibitor; and a TEC kinase inhibitor.

In certain embodiments, the co-former is a sphingosine 1-phosphate (SIP) receptor modulator.

In certain embodiments, the co-former is etrasimod or ozanimod.

In certain embodiments, the co-former is a steroidal anti-inflammatory agent. As a non-limiting example, the co-former can be beclomethasone 17 or budesonide.

In certain embodiments, the co-former is a non-steroidal anti-inflammatory agent such as 5-ASA.

In certain embodiments, the co-former is a receptor-interacting protein kinase 1 (RIPK1) inhibitor such as GSK2982772.

In certain embodiments, the co-former is an EP4 modulator such as KAG-308.

In certain embodiments, the co-former is a toll-like receptor (e.g., TLR4, TLR9) modulator. In certain of these embodiments, the co-former is a TLR4 modulator such as JKB-122. In certain embodiments, the co-former is a TLR9 modulator such as cobitolimod.

In certain embodiments, the co-former is a Janus kinase (JAK) inhibitor. In certain of these embodiments, the co-former is selected from the group consisting of TD-1473, tofacitinib, upadacitinib, filgotinib, PF-06651600, and PF-06700841.

In certain embodiments, the co-former is a lanthionine synthetase C-like 2 (LANCL2) modulator such as BT-11.

In certain embodiments, the co-former is a phosphatidylcholine such as LT-02.

In certain embodiments, the co-former is an integrin modulator. In certain of these embodiments, the co-former is an α4 Integrin modulator such as AJM-300 (carotegrast).

In certain embodiments, the co-former is a Smad7 antisense oligonucleotide such as mongersen.

In certain embodiments, the co-former is a phosphodiesterase 4 (PDE4) modulator such as apremilast.

In certain embodiments, the co-former is a tumor progression locus 2 (TPL2) inhibitor such as GS-4875.

In certain embodiments, the co-former is a tyrosine kinase 2 (TYK2) inhibitor.

In certain of these embodiments, the co-former is BMS-986165, PF-06700841, or PF-06826647.

In certain embodiments, the co-former is a TEC kinase inhibitor such as PF-06651600.

Non-Limiting Combinations

In some embodiments, the cocrystal includes (i) niclosamide; and (ii) a pharmaceutically acceptable salt of niclosamide; or a pharmaceutically acceptable salt and/or hydrate of niclosamide of a niclosamide analog.

In some embodiments, the cocrystal includes (i) niclosamide; and (ii) a second API.

In some embodiments, the cocrystal includes (i) a pharmaceutically acceptable salt of niclosamide; and (ii) a second API.

In some embodiments, the cocrystal includes (i) niclosamide; and (ii) a second API.

In some embodiments, the cocrystal includes (i) a pharmaceutically acceptable salt of niclosamide; and (ii) an amino acid (e.g., proline, e.g., D-proline, or L-proline, or racemic proline).

In some embodiments, the cocrystal includes (i) niclosamide; and (ii) an amino acid (e.g., proline, e.g., D-proline, or L-proline, or racemic proline).

In some embodiments, the cocrystal includes (i) a pharmaceutically acceptable salt of niclosamide; and (ii) a 5-10 (e.g., 5-9, 5-6, or 5) membered heteroaryl, e.g., a nitrogen-containing heteroaryl, e.g., imidazole.

In some embodiments, the cocrystal includes (i) niclosamide; and (ii) a 5-10 (e.g., 5-9, 5-6, or 5) membered heteroaryl, e.g., a nitrogen-containing heteroaryl, e.g., imidazole.

For examples, see Sanphui, P. Cryst. Growth Des. 2012, 12, 4588; Imramovský, A. Crystals 2012, 2, 349-361; and Grifasi, F. Cryst. Growth Des. 2015, 15, 4588.

Niclosamide Compound of the Co-Crystal

In some embodiments, the chemical purity of the niclosamide compound can be as defined anywhere herein.

Particle Size of the Co-Crystal

In some embodiments, the co-crystal can have a reduced particle size as defined anywhere herein for the niclosamide compounds.

In some embodiments, co-crystals having reduced particle size can be prepared by jet milling, e.g., using CMTI equipment NGMP-Mill-A, a 2-inch, pancake micronizer manufactured by Sturtevant.

Particle Size Distribution (PSD) can be determined by laser diffraction technique, e.g., using a “MALVERN MASTERSIZER 2000” (standard range between 0.020 and 2000.0 microns), model “APA 2000”, equipped with “Hydro 2000 sm” as dispersing unit.

In some embodiments, the co-crystal has a reduced particle size range.

In some embodiments, co-crystal has a particle size range of from about 0.1 μm to about 30 μm. In certain embodiments, the co-crystal has a particle size range of from about 0.1 to about 20 μm. In certain embodiments, the co-crystal has a particle size range of from about 0.1 μm to about 10 μm.

In some embodiments, the co-crystal has a particle size distribution D(0.9) of from about 1.0 μm to about 15.0 μm. In certain embodiments, the co-crystal has a particle size distribution D(0.9) of from about 1.0 μm to about 10.0 μm. In certain embodiments, the co-crystal has a particle size distribution D(0.9) of from about 6.0 μm to about 8.0 μm. In certain embodiments, the co-crystal has a particle size distribution D(0.9) of from about 2.2 μm to about 3.2 μm.

In some embodiments, the co-crystal has a particle size distribution D(0.1) of from about 0.1 μm to about 1.5 μm. In certain embodiments, the co-crystal has a particle size distribution D(0.1) of from about 0.1 μm to about 1.0 μm. In certain embodiments, the co-crystal has a particle size distribution D(0.1) of from about 0.3 μm to about 0.9 μm.

In some embodiments, the co-crystal has a particle size distribution D(0.5) of from about 0.5 μm to about 6.0 μm. In certain embodiments, the co-crystal has a particle size distribution D(0.5) of from about 1.0 μm to about 4.0 μm. In certain embodiments, the co-crystal has a particle size distribution D(0.5) of from about 1.0 μm to about 2.0 μm. In certain embodiments, the co-crystal has a particle size distribution D(0.5) of from about 2.5 μm to about 3.5 μm.

In some embodiments, the co-crystal has a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the co-crystal has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the co-crystal has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the niclosamide compound has a chemical purity of greater than about 99.0%; and the co-crystal has a particle size distribution D(0.9) of from about 1.0 to about 10.0 μm, a particle size distribution D(0.5) of from about 1.0 μm to about 4.0 μm, and a particle size distribution D(0.1) of from about 0.1 μm to about 1.0 μm.

In some embodiments, the niclosamide compound has a chemical purity of greater than about 99.0%; and the co-crystal has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the niclosamide compound has a chemical purity of greater than about 99.0%; and the co-crystal has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the niclosamide compound has a chemical purity of greater than about 99.0%; and the co-crystal has a particle size range of from about 0.1 μm to about 30 μm, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the niclosamide compound has a chemical purity of greater than about 99.0%; and the co-crystal has a particle size range of from about 0.1 μm to about 30 μm, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the niclosamide compound has a chemical purity of greater than about 99.0%; and the co-crystal has a particle size range of from about 0.1 μm to about 30 μm, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In certain of the foregoing embodiments, the co-crystal has a particle size distribution D(0.5) of from about 2.5 μm to about 3.5 μm.

In certain other of the foregoing embodiments, the co-crystal has a particle size distribution D(0.5) of from about 1.0 μm to about 2.0 μm.

Pharmaceutical Compositions and Administration

General

A niclosamide compound, or a pharmaceutically acceptable salt and/or cocrystal thereof; e.g., a compound, such as niclosamide, or a pharmaceutically acceptable salt and/or cocrystal thereof) is administered to a subject in need thereof by any route which makes the compound bioavailable (e.g., locally bioavailable). In certain embodiments, the route is respiratory administration.

In some embodiments, a niclosamide compound, or a pharmaceutically acceptable salt and/or cocrystal thereof; e.g., a compound, such as niclosamide, or a pharmaceutically acceptable salt and/or cocrystal thereof) is administered as a pharmaceutical composition that includes the chemical entity and one or more pharmaceutically acceptable excipients, and optionally one or more other therapeutic agents as described herein.

In some embodiments, the niclosamide compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium tri silicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the compositions and methods described here can further include bicarbonate. While not wishing to be bound by theory, it is believed that the inclusion of bicarbonate can modulate (e.g., potentiate) the activity of a niclosamide compound (or a pharmaceutically acceptable salt thereof), e.g., niclosamide and/or one or more second therapeutic agents co-administered with the niclosamide compound (e.g., niclosamide). See, e.g, WO 2018/141063 which is incorporated herein by reference in its entirety.

Accordingly, in another aspect, any of the methods described herein can include contacting a coronavirus (e.g., the coronavirus leading to COVID 19) with an effective amount of a bicarbonate (e.g., sodium bicarbonate, ammonium bicarbonate, lithium bicarbonate, potassium bicarbonate, magnesium bicarbonate, calcium bicarbonate or zinc bicarbonate) and an effective amount of a niclosamide compound (or a pharmaceutically acceptable salt thereof), e.g., niclosamide.

In another aspect, any of the methods described herein can include administering to a subject (e.g., a subject having or at risk of having coronavirus infection, e.g., COVID-19) an effective amount of a bicarbonate (e.g., sodium bicarbonate, ammonium bicarbonate, lithium bicarbonate, potassium bicarbonate, magnesium bicarbonate, calcium bicarbonate or zinc bicarbonate) and an effective amount of a niclosamide compound (or a pharmaceutically acceptable salt thereof), e.g., niclosamide. and.

Bicarbonate forms the dominant buffering system in the human body, which plays an important role in maintaining the pH of blood around 7.4.

In some embodiments, the bicarbonate is potassium, lithium, calcium, magnesium, sodium, ammonium or zinc bicarbonate. In some embodiments, the bicarbonate is sodium bicarbonate or ammonium bicarbonate. In some embodiments, the bicarbonate is sodium bicarbonate.

In some embodiments, the dosage or amount of the bicarbonate can be an amount to provide physiological concentrations of bicarbonate, or about 25 mM of bicarbonate. In some embodiments, the bicarbonate can be provided in a composition comprising about 1 mM to about 150 mM bicarbonate, or about 20 mM to about 50 mM bicarbonate.

In some embodiments, the bicarbonate can be provided in a composition comprising about 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or even about 150 mM bicarbonate.

In some embodiments, the bicarbonate can be provided in a composition comprising about 0.01 wt % to about 1.0 wt % bicarbonate, or about 0.20 wt % to about 0.5 wt % bicarbonate. In some embodiments, the effective amount or dosage of bicarbonate can be about 0.01 mg to about 1 mg per kg of body weight of the subject. In some embodiments, the bicarbonate can be provided in a composition comprising about 0.01 mg to about 1 mg of bicarbonate.

In some embodiments, the amount of the bicarbonate is a physiological concentration of the bicarbonate. In some embodiments, the bicarbonate is present in a composition having a concentration of about 1 mM to about 150 mM of bicarbonate. In some aspects, the bicarbonate is present in a composition being about 0.01 wt % to about 1 wt % of bicarbonate.

In some embodiments, bicarbonate useful in the methods and compositions described herein is a component of a buffer. In certain embodiments, the bicarbonate and the niclosamide compound (e.g., niclosamide) are present in the same pharmaceutical composition. In certain other embodiments, the bicarbonate and the niclosamide compound are not present in the same pharmaceutical composition. In some embodiments, the bicarbonate can be administered at about the same time (e.g., concurrently) as, e.g., a niclosamide compound, e.g., niclosamide, and/or one or more second therapeutic agents, or at a different time (e.g., sequentially).

In some embodiments, the niclosamide compounds described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intraci sternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumor, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Local Administration

In some embodiments, the niclosamide compounds described herein or a pharmaceutical composition thereof are suitable for local administration, e.g., local administration by way of administering the niclosamide compounds or composition thereof at a particular treatment site, (e.g., the respiratory tract, e.g., the upper respiratory tract (e.g., nose or nasal passage) or lower respiratory tract (e.g., lungs); e.g., the digestive tract, the gastrointestinal (“GI”) tract, e.g., colon; e.g., eye, e.g., skin, e.g., endothelial cells (e.g., blood vessels)) so as to provide local administration of the chemical entity to the area in need of treatment (e.g., respiratory tract (e.g., nasal passage or the lungs) or the digestive tract (e.g., colon); eye, skin). In certain embodiments, relatively low systemic exposure of the niclosamide compounds occurs during said local administration. Examples of such compositions include, e.g., compositions suitable for administration by inhalation.

In some embodiments, the niclosamide compound described herein or a pharmaceutical composition thereof are suitable for local administration to the respiratory tract, e.g., the upper respiratory tract (e.g., nose or nasal passage) or lower respiratory tract (e.g., lungs). In certain embodiments, upon administration, the local concentration of the niclosamide compound in the respiratory tract is higher (e.g., from about 2 times higher to about 1,000 times higher; from about 2 times higher to about 900 times higher; from about 2 times higher to about 800 times higher; from about 2 times higher to about 700 times higher; from about 2 times higher to about 500 times higher; from about 2 times higher to about 400 times higher; from about 2 times higher to about 300 times higher; from about 2 times higher to about 200 times higher; from about 2 times higher to about 100 times higher; from about 2 times higher to about 50 times higher, from about 5 times higher to about 1,000 times higher; from about 5 times higher to about 900 times higher; from about 5 times higher to about 800 times higher; from about 2 times higher to about 700 times higher; from about 5 times higher to about 500 times higher; from about 5 times higher to about 400 times higher; from about 5 times higher to about 300 times higher; from about 5 times higher to about 200 times higher; from about 5 times higher to about 100 times higher; from about 5 times higher to about 50 times higher; from about 5 times higher to about 25 times higher; from about 5 times higher to about 15 times higher; e.g., about 1,000 times higher, about 900 times higher, about 800 times higher, about 700 times higher, about 600 times higher, about 500 times higher, about 400 times higher, about 300 times higher, about 200 times higher, about 100 times higher, about 50 times higher, about 25 time higher, about 20 times higher, about 15 times higher, about 10 times higher, about 5 times higher) than the concentration of the chemical entity in the plasma compartment. In certain of these embodiments, the chemical entity in the plasma compartment is subject to first pass metabolism.

In some embodiments, the niclosamide compound described herein or a pharmaceutical composition thereof are suitable for local administration to one or more specific locations within the respiratory tract. For example, at least some of the niclosamide compound is present in the upper respiratory tract (e.g., nose and nasal passages, paranasal sinuses, the pharynx, and the portion of the larynx above the vocal folds (cords) (e.g., nose and nasal passages); or at least some of the niclosamide compound is present in the lower respiratory tract (e.g., portion of the larynx below the vocal folds, trachea, bronchi, and lungs (e.g., lungs)). Methods of said local administration can include, without limitation, respiratory administration such as inhalation or intranasal administration.

In some embodiments, the niclosamide compound described herein or a pharmaceutical composition thereof are suitable for local administration to the GI tract, e.g., colon. In certain embodiments, upon administration, the local concentration of the niclosamide compound in the GI tract is higher (e.g., from about 2 times higher to about 1,000 times higher; from about 2 times higher to about 900 times higher; from about 2 times higher to about 800 times higher; from about 2 times higher to about 700 times higher; from about 2 times higher to about 500 times higher; from about 2 times higher to about 400 times higher; from about 2 times higher to about 300 times higher; from about 2 times higher to about 200 times higher; from about 2 times higher to about 100 times higher; from about 2 times higher to about 50 times higher, from about 5 times higher to about 1,000 times higher; from about 5 times higher to about 900 times higher; from about 5 times higher to about 800 times higher; from about 2 times higher to about 700 times higher; from about 5 times higher to about 500 times higher; from about 5 times higher to about 400 times higher; from about 5 times higher to about 300 times higher; from about 5 times higher to about 200 times higher; from about 5 times higher to about 100 times higher; from about 5 times higher to about 50 times higher; from about 5 times higher to about 25 times higher; from about 5 times higher to about 15 times higher; e.g., about 1,000 times higher, about 900 times higher, about 800 times higher, about 700 times higher, about 600 times higher, about 500 times higher, about 400 times higher, about 300 times higher, about 200 times higher, about 100 times higher, about 50 times higher, about 25 time higher, about 20 times higher, about 15 times higher, about 10 times higher, about 5 times higher) than the concentration of the chemical entity in the plasma compartment. In certain of these embodiments, the chemical entity in the plasma compartment is subject to first pass metabolism.

In some embodiments, the niclosamide compound described herein or a pharmaceutical composition thereof are suitable for local administration to one or more specific locations within the digestive or GI tract, e.g., colon. For example, at least some of the niclosamide compound is present in the upper GI tract (e.g., stomach); or at least some of the niclosamide compound is present in the lower GI tract (e.g., the large intestine, e.g., the colon, e.g., the ascending colon and/or transverse colon and/or distal colon; or the small bowel). As a further example, at least some of the niclosamide compound is present in the ascending colon and/or the transverse colon and/or the distal colon and/or the small bowel and/or the stomach. Methods of said local administration can include, without limitation, oral administration and/or rectal administration.

In one aspect, provided herein is a composition comprising a niclosamide compound or co-crystal as described anywhere herein and one or more pharmaceutically acceptable excipients, wherein the composition is suitable for oral administration.

In one aspect, provided herein is a composition comprising a niclosamide compound or co-crystal as described anywhere herein and one or more pharmaceutically acceptable excipients, wherein the composition is suitable for local, topical administration. In certain embodiments, the chemical entities described herein or a pharmaceutical composition thereof are suitable for rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In some embodiments, administration of a single dose of the composition to a subject produces a local concentration of the niclosamide compound in the GI tract (e.g., colon) of the subject that is higher than the concentration of the compound in the plasma compartment of the subject.

In some embodiments, administration of a single dose of the composition to a subject produces a local concentration of the niclosamide compound in the GI tract (e.g., colon) of the subject that is at least about 200 times higher than the concentration of the compound in the plasma compartment of the subject.

In some embodiments, administration of a single dose of the composition to a subject produces a local concentration of the niclosamide compound in the GI tract (e.g., colon) of the subject that is at least about 300 times higher than the concentration of the compound in the plasma compartment of the subject.

In some embodiments, administration of a single dose of the composition to a subject produces a local concentration of the niclosamide compound in the GI tract (e.g., colon) of the subject that is at least about 500 times higher than the concentration of the compound in the plasma compartment of the subject.

In some embodiments, administration of a single dose of the composition to a subject produces a local concentration of the niclosamide compound in the GI tract (e.g., colon) of the subject that is at least about 700 times higher than the concentration of the compound in the plasma compartment of the subject.

In some embodiments, the local concentration of the niclosamide compound in the GI tract (e.g., colon) of the subject is higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 2 times higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 5 times higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 10 times higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 25 times higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 50 times higher than a local concentration produced by oral administration of a single dose of a second composition comprising a second niclosamide compound, wherein the second niclosamide compound has a higher particle size than the first niclosamide compound.

In some embodiments, the local concentration of the niclosamide compound in the GI tract (e.g., colon) of the subject is at least about 100 times higher than a local concentration produced by oral administration of a single dose of a second composition comprising a second niclosamide compound, wherein the second niclosamide compound has a higher particle size than the first niclosamide compound.

In some embodiments, the second niclosamide compound has a particle size distribution D(0.9) of from about 25.0 μm to about 65.0 μm.

In some embodiments, the second niclosamide compound has a particle size distribution D(0.1) of from about 4.0 μm to about 10.0 μm.

In another aspect, provided herein is a dosage form (e.g., a unit dosage form) comprising a composition as described anywhere herein, wherein the dosage form is suitable for oral administration.

In another aspect, provided herein is a dosage form (e.g., a unit dosage form) comprising a composition as described anywhere herein, wherein the dosage form is suitable for rectal administration.

In some embodiments, the dosage form further comprises one or more components that chemically and/or structurally predispose the dosage form for delivery of the compound to the ascending colon.

In some embodiments, the dosage form further comprises one or more components that chemically and/or structurally predispose the dosage form for delivery of the compound to the transverse colon.

In some embodiments, the dosage form further comprises one or more components that chemically and/or structurally predispose the dosage form for delivery of the compound to the distal colon.

In some embodiments, the dosage form further comprises one or more components that chemically and/or structurally predispose the dosage form for delivery of the compound to the small bowel.

In one aspect, provided herein is a composition comprising a niclosamide compound or co-crystal as described anywhere herein and one or more pharmaceutically acceptable excipients, wherein the composition is suitable for respiratory administration (e.g., inhalation).

In some embodiments, administration of a single dose of the composition to a subject produces a local concentration of the niclosamide compound in the lower respiratory tract (e.g., lungs) of the subject that is higher than the concentration of the compound in the upper respiratory tract of the subject.

Inhalation and Intranasal Therapy

In some embodiments, niclosamide or a pharmaceutically acceptable salt thereof can be formulated into any suitable dosage form. Non-limiting examples of such dosage forms include aerosols, dispersions (e.g., aqueous oral dispersions, self-emulsifying dispersions, liposomal dispersions, dispersions with colloidal silica or nanospheres such as hydroxypropylmethylcellulose phthalate (HPMCP) nanospheres), pegylated liposomes, liquids, elixirs, suspensions (e.g., nanosuspensions), aerosols, controlled release formulations, lyophilized formulations, powders, delayed release formulations, extended release formulations, multiparticulate formulations, and mixed immediate release formulations. In some embodiments, niclosamide or a pharmaceutically acceptable salt thereof, can be formulated for administration intranasally and/or by inhalation, e.g., using an inhalation device.

An “inhalation device,” as used herein, refers to any device that is capable of administering a drug formulation to the respiratory airways of a subject. Inhalation devices include conventional inhalation devices such as nebulizers, metered dose inhalers (MDIs), dry powder inhalers (DPIs), heat vaporizers, soft mist inhalers, thermal aerosol inhalers, or electrohydrodynamic-based solution misting inhalers. Inhalation devices also include nebulizers. “Nebulizer,” as used herein, refers to a device that turns medications, compositions, formulations, suspensions, and mixtures, etc. into a fine aerosol mist for delivery to the lungs. Non-limiting examples of nebulizers include jet nebulizers, mesh nebulizers, and ultrasonic wave nebulizers. Nebulizers, metered dose inhalers, and soft mist inhalers deliver pharmaceuticals by forming an aerosol which includes droplet sizes that can easily be inhaled. The aerosol can be used by a subject within the bounds of an inhalation therapy, whereby the niclosamide or a pharmaceutically-acceptable salt thereof reaches the subject's respiratory tract upon inhalation. In some embodiments, the methods disclosed herein comprise administering to a subject a nominal dose of niclosamide or a pharmaceutically-acceptable salt thereof by an inhalation device, such as a nebulizer.

In some embodiments of the methods disclosed herein, administration of a composition comprising niclosamide or a pharmaceutically acceptable salt thereof, to a subject with an inhalation device, e.g., a nebulizer, a metered dose inhaler, a dry powder inhaler (DPI), a jet nebulizer, an ultrasonic wave nebulizer, a heat vaporizer, a soft mist inhaler, a thermal aerosol inhaler, or an electrohydrodynamic-based solution misting inhaler, is effective for the treatment or prophylaxis of long hauler's syndrome associated with COVID-19 in a subject.

Inhalation devices may be mechanical or electrical, and include, for example, jet nebulizers and ultrasonic nebulizers. Jet nebulizers generally utilize compressors to generate compressed air, which breaks the liquid medication into small breathable droplets, which form an aerosolized (atomized) mist. In some embodiments, when the subject breathes in, a valve at the top opens, which then allows air into the apparatus, thereby increasing the rate of mist generation; when the subject breathes out, the top valve closes, thereby slowing down mist generation while simultaneously permitting the subject to breathe out through the opening of a mouthpiece flap. Some nebulizers may provide the aerosol in a continuous mode (e.g., the eFlow from PARI Pharma Starnberg), by a breath enhanced mode (e.g., the PART LC Plus or Sprint from PARI Starnberg), by breath actuated mode dependent on the breathing pattern of the subject (e.g., the AeroEclipse from Trudell, Canada or the I-Neb from Philips Respironics), or according to given inhalation profile (e.g., the Akita from Activaero, Gmuenden, Germany).

Some conventional inhalation devices are disclosed in U.S. Pat. Nos. 9,566,399, 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, and International Publication Nos. WO 2018/191776 and WO 2018/213834, each of which is hereby incorporated by reference in its entirety. Commercial conventional inhalation devices are available from: PARI (Germany) under the trade names PARI LC Plus®, LC Star®, and PARI-Jet®; A & H Products, Inc. (Tulsa, Okla.) under the trade name AquaTower®; Hudson RCI (Temecula, Calif.) under the trade name AVA-NEB®; Intersurgical, Inc. (Liverpool, N.Y.) under the trade name Cirrus®; Salter Labs (Arvin, Calif.) under the trade name Salter 8900®; Respironics (Murrysville, Pa.) under the trade name Sidestream®; Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®; or Trudell, Canada under the trade name AeroEclipse®.

In some embodiments of the methods disclosed herein, compositions comprising niclosamide or a pharmaceutically acceptable salt thereof are administered with a dry powder inhaler. Compositions administered with dry powder inhalers can include nanoparticles, spray dried materials, engineered porous particles with low mass median diameter but a high geometric diameter, liposomes, stealth (or PEGylated) liposomes, or combinations thereof. In some embodiments, compositions administered by dry powder inhalers administered in the methods disclosed herein comprise nanoparticle clusters that aggregate into micrometer sized particles at neutral or basic pH but dissociate into nanoparticles at the pH encountered in the lung. In some embodiments the nanoparticle clusters comprise fumaryl diketopiperazine. In some embodiments, compositions administered with dry powder inhalers comprise lactose.

In some embodiments, a dry powder inhaler used to administer an inhalation formulation in the methods disclosed herein comprises a pre-metered dose. For example a pre-metered dose inhaler can comprise a capsule pre-filled with a powder (e.g., a Plastiape Monodose inhaler). In some embodiments, a dry powder inhaler used to administer an inhalation formulation in the methods disclosed herein has a device-metered system such as Twisthaler, sold by Schering Plough, which comprises a reservoir to store a powder and a twisting top to dispense each dose. Inhalation formulations for administration with a dry powder inhaler may be prepared by blending niclosamide or a pharmaceutically acceptable salt thereof, with lactose, or spray drying niclosamide or a pharmaceutically acceptable salt thereof, or by pelletizing niclosamide or a pharmaceutically acceptable salt thereof, to form free-flowing spherical agglomerates.

In some embodiments of the methods disclosed herein, compositions comprising niclosamide or a pharmaceutically acceptable salt thereof are administered with a metered dose inhaler. In some embodiments, a composition administered with a metered dose inhaler in the methods disclosed herein comprises one or more of nanoparticles, spray dried materials, engineered porous particles with low mass median diameter but a high geometric diameter, liposomes, and stealth (or PEGylated) liposomes.

In some embodiments of the methods disclosed herein, compositions comprising niclosamide or a pharmaceutically acceptable salt thereof are administered with a thermal aerosol inhaler.

In some embodiments of the methods disclosed herein, compositions comprising niclosamide or a pharmaceutically acceptable salt thereof are administered with an electrohydrodynamic-based solution misting inhaler. In some embodiments, the aerosol in the electrohydrodynamic-based solution-misting inhaler is generated by subjecting a solution of niclosamide or a pharmaceutically acceptable salt thereof, or a liposome or pegylated liposome comprising niclosamide or a pharmaceutically acceptable salt thereof, to electrohydrodynamic forces through electrostatic energy.

Nebulizers are inhalation devices that comprise a micro-perforated membrane through which a liquid solution is converted through electrical or mechanical means into aerosol droplets suitable for inhalation. Nebulizers can deliver a large fraction of a loaded dose to a subject. In some embodiments, the nebulizer also utilizes one or more actively or passively vibrating microperforated membranes. In some embodiments, the nebulizer contains one or more oscillating membranes. In some embodiments, the nebulizer contains a vibrating mesh or plate with multiple apertures and optionally a vibration generator with an aerosol mixing chamber. In some such embodiments, the mixing chamber functions to collect (or stage) the aerosol from the aerosol generator. In some embodiments, an inhalation valve is also used to allow an inflow of ambient air into the mixing chamber during an inhalation phase and is closed to prevent escape of the aerosol from the mixing chamber during an exhalation phase. In some such embodiments, the exhalation valve is arranged at a mouthpiece which is removably mounted at the mixing chamber and through which the subject inhales the aerosol from the mixing chamber. Still yet, in some embodiments, the nebulizer contains a pulsating membrane. In some embodiments, the nebulizer is continuously operating.

In some embodiments, the nebulizer contains a vibrating micro-perforated membrane of tapered nozzles that generates a plume of droplets without the need for compressed gas. In these embodiments, a solution in the micro-perforated membrane nebulizer is in contact with a membrane, the opposite side of which is open to the air. The membrane is perforated by a large number of nozzle orifices of an atomizing head. An aerosol is created when alternating acoustic pressure in the solution is built up in the vicinity of the membrane causing the fluid on the liquid side of the membrane to be emitted through the nozzles as uniformly sized droplets.

Some embodiments of nebulizers use passive nozzle membranes and a separate piezoelectric transducer that stimulates the membrane. In contrast, some nebulizers employ an active nozzle membrane, which use the acoustic pressure in the nebulizer to generate very fine droplets of solution via the high frequency vibration of the nozzle membrane.

Some nebulizers can contain a resonant system. For example, in such nebulizers, the membrane is driven by a frequency for which the amplitude of the vibrational movement at the center of the membrane is particularly large, resulting in a focused acoustic pressure in the vicinity of the nozzle; the resonant frequency may be about 100 kHz. A flexible mounting is used to keep unwanted loss of vibrational energy to the mechanical surroundings of the atomizing head to a minimum. In some embodiments, the vibrating membrane of the nebulizer may be made stainless steel, or of a nickel-palladium alloy by electroforming.

In some embodiments, a nebulizer may be adapted or adaptable to operate in conjunction with a unit dosage form, such as an ampule or vial, which contains a single dose of a composition comprising niclosamide, or a pharmaceutically-acceptable salt thereof, for the treatment of long hauler's syndrome associated with COVID-19. The unit dosage form comprises a container that contains an inhalation formulation comprising the niclosamide, or a pharmaceutically-acceptable salt thereof. The container is adapted to cooperate with the nebulizer device in such a way as to permit administration of the nominal dose of the inhalation formulation to a subject. In some embodiments, the nebulizer and the unit dosage form are configured so that they are useable together, but not with other devices or dosage forms. In some particular embodiments, the unit dosage form is configured such that it fits into a keyhole-like structure in the nebulizer, but will not operate with other nebulizer devices. In such embodiments, the nebulizer is configured such that it will accept and properly operate with the unit dosage form containing the niclosamide, or a pharmaceutically-acceptable salt thereof, but not with other dosage forms.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif.) under the trade names I-Neb®; Omron (Bannockburn, Ill.) under the trade name Micro-Air®; Activaero (Germany) under the trade name Akita®, and AerovectRx (Atlanta, Ga.) under the trade name AerovectRx®.

In some embodiments, a composition comprising niclosamide, or a pharmaceutically acceptable salt thereof, is formulated as an inhalable nanosuspension (see, e.g., Costabile et al. Mol Pharm. 2015 Aug. 3; 12(8):2604-17.) In some embodiments, an inhalable nanosuspension of niclosamide, or a pharmaceutically acceptable salt thereof, is administered to a subject using a nebulizer.

In some embodiments, devices for intranasal administration of niclosamide, or a pharmaceutically acceptable salt thereof, include one or more features present in any inhalation device described herein. In some embodiments, devices for intranasal administration of niclosamide, or a pharmaceutically acceptable salt thereof, are spray devices. Suitable commercially available nasal spray devices include Accuspray™ (Becton Dickinson). In some embodiments, spray devices for intranasal use are devices for which the performance of the device is not dependent upon the pressure applied by the user. These devices are known as pressure threshold devices. Pressure threshold devices release liquid from the nozzle only when a threshold pressure is applied. These devices make it easier to achieve a spray with a regular droplet size. Pressure threshold devices suitable for use with the present invention are known in the art and are described for example in WO 91/13281, EP 311863, and EP 516636. Pressure threshold devices are commercially available from Pfeiffer GmbH and are also described in Bommer, R. Pharmaceutical Technology Europe, September 1999.

In some embodiments, the intranasal devices can administer niclosamide, or a pharmaceutically acceptable salt thereof, by means of bi-dose delivery. Bi-dose devices contain two sub-doses of a single dose, one sub-dose for administration to each nostril. Generally, the two sub-doses are present in a single chamber and the construction of the device allows for efficient delivery of a single sub-dose at a time. Alternatively, a monodose device may be used for administering the vaccines according to the invention.

In some embodiments, niclosamide, or a pharmaceutically acceptable salt thereof, is formulated as an ointment or gel for intranasal delivery.

In some embodiments, the compositions disclosed herein can include one or pharmaceutical excipients that provide suitable properties for intranasal administration and/or administration by inhalation of niclosamide, or a pharmaceutically acceptable salt thereof. See, e.g., Labiris and Dolovich, Br J Chn Pharmacol. 2003 December; 56(6): 600-612. Non-limiting examples of such pharmaceutical excipients can include surfactants, suspending agents, viscosity enhancing agents, wetting agents, and propellants.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, poloxomers, bile salts, glyceryl monostearate, copolymers of ethylene oxide and propylene oxide, e.g., Pluronic® (BASF), and the like. Additional examples of surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium docusate, triacetin, Tween 80, vitamin E TPGS, ammonium salts and the like.

Non-limiting examples of propellants include chlorofluorocarbons (CFC) and hydrofluoroalkanes (HFAs).

Compositions and formulations of the disclosure may have a surface tension effective for deposition, penetration or retention of the composition primarily in the peripheral lung regions, including the bronchioles and alveoli.

The niclosamide compounds described herein (e.g., niclosamide) can be formulated as a brittle matrix particle composition, comprising brittle matrix particles of niclosamide compounds which upon pulmonary delivery can be fractured to release primary particles of niclosamide or aggregates of the primary particles of niclosamide. In certain embodiments, the primary particles or aggregates of the primary particles have an aerodynamic diameter of between 2 and 5 microns. The brittle matrix particles can comprise porous matrix of nanostructured primary particles of niclosamide compounds (e.g., niclosamide). Said matrix of nano-structured primary particles can be formed e.g., by dissolving the niclosamide compound in a solvent to form a niclosamide/solvent mixture, freezing (e.g., rapidly freezing) niclosamide/solvent (e.g., on a cryogenically cooled surface) and removing the solvent from the niclosamide/solvent mixture. The nano-structured matrix of primary particles can be fractured (e.g., through device-induced shearing) to release primary particles or aggregates of the primary particles, both of which are smaller than the matrix of nano-structured particles. The fractured particles are suitable e.g., for deep lung delivery. Methods of making and using brittle matrix particles are described in U.S. Pat. Nos. 10,092,512; 10,434,062; US 2009/0208582; US 2020/0069572; and WO 2009/103035, each of which is incorporated herein by reference in its entirety.

Brittle matrix particles can have a higher surface area compared to particles prepared with other techniques such as jet milling or physical mixtures. In some aspects, the niclosamide brittle matrix particles have a specific surface area of greater than 5 m²/g. The brittle matrix particles may have a specific surface area from about 5 m²/g to about 1000 m²/g, from about 10 m²/g to about 500 m²/g, or from about 20 m²/g to about 250 m²/g. In some embodiments, the specific surface area is from about 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, to about 1000 m²/g, or any range derivable therein. Surface area of brittle matrix particles can be calculated e.g., using methods described in WO 2016/178704, which is incorporated herein by reference in its entirety.

The brittle matrix particles can be asymmetric particles (e.g., anisotropic particles). For example, the brittle matrix particles can be rods (e.g., formed by thin-film freezing). In some embodiments, the brittle matrix particle compositions herein comprise one or more anisotropic particles with an aspect ratio of about 0.02, 0.05, 0.1, 0.2, 0.3, 0.4, 0.5, 0.6, 0.7, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 5, 10 20, and 50. As a non-limiting example, the one or more anisotropic particles may have an aspect ratio range of between 0.1 and 2.0 or greater, e.g., the aspect ratio may be 0.2, 0.3, 0.4, 0.5, 0.6, 0.7, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 and so on. In certain embodiments, the aspect ratios are greater than 1.

The brittle matrix particle compositions comprising niclosamide compounds described herein (e.g., niclosamide) can have a total emitted dose (or emitted dose) of greater than 80% of the active pharmaceutical ingredient. In certain embodiments, the total emitted dose is from about 80% to about 100%, from about 85% to about 100%, or from about 90% to about 100%. The total emitted dose can be calculated e.g., using methods described in WO 2016/178704, which is incorporated herein by reference in its entirety. In certain embodiments, the brittle matrix particle compositions produce an FPF value of over 50% (e.g., over 55%, over 60%, over 65%, or over 70%).

Brittle matrix particles can be prepared by mixing the niclosamide compounds described herein (e.g., niclosamide) into a solvent (e.g., solvent system comprising water and an organic solvent) to provide a mixture and freezing the mixture using methods known in the art such as ultra-rapid freezing, spray freeze drying, or thin film freezing. Exemplary methods of freezing include those described in US 2010/0221343; U.S. Pat. No. 10,092,512; US 2004/0022861; U.S. Pat. No. 6,862,890; WO 2002/060411; U.S. Pat. No. 9,175,906; US 2016/0074328; WO 2004/064808; WO 2016/178704; and Respirable Low-Density Microparticles Formed In Situ from Aerosolized Brittle Matrices, Pharmaceutical Research, 30(3):813-825, 2013, each of which is incorporated herein by reference in its entirety. In certain embodiments, the brittle matrix particles are prepared using spray freeze drying (SFD) or thin film freezing (TFF) e.g., as described in US 2010/0221343 and Respirable Low-Density Microparticles Formed In Situ from Aerosolized Brittle Matrices, Pharmaceutical Research, 30(3):813-825, 2013 (supra). After freezing, the brittle matrix particles may be further subjected to drying to obtain a dry powder (e.g., dry powder suitable for aerosol administration). As a non-limiting example, the brittle matrix particles may be dried through lyophilization and other methods known to those of skill in the art. Without wishing to be bound by any theory, the brittle matrix particles and the fast freezing drying methods allow the mixing of the particles while maintaining the homogeneity of the mixture wile preventing segregation of the different components. The improved homogeneity may also be exhibited during the aerosolization process. In some embodiments, the brittle matrix particles are prepared using thin film freezing (TFF) methods. In some embodiments, the methods comprise dissolving the pharmaceutical composition in a solvent. Some solvents which may be used in the methods described herein include water, an organic solvent, or a mixture thereof. The organic solvents that may be used herein include polar organic solvents such an alcohol, a heterocyclic compound, an alkylnitrile, or a mixture thereof. Some non-limiting examples of polar organic solvents include methanol, ethanol, isopropanol, tert-butanol (tertiary butanol), dimethylsulfoxide, dimethylformamide, 1,4-dioxane, or acetonitrile. In some embodiments, mixtures of these solvents are contemplated. Such mixtures may comprise one or more organic solvents with water. One non-limiting example of these mixtures includes the solvent mixture of tertbutanol, 1,4-dioxane, acetonitrile, and water. The solvent mixture may comprise a mixture of tertiary butanol, 1,4-dioxane, acetonitrile, and purified water in a ratio of 2:1:3:3 (v/v).

In some embodiments, a brittle matrix particle composition of the niclosamide compounds herein (e.g., niclosamide) comprise brittle matrix particles of the niclosamide compounds without an excipient. In some other embodiments, thin-film freezing methods may be used in a manner to allow for the co-deposition of niclosamide and one or more excipients to form a pharmaceutical composition. Accordingly, in some embodiments, the brittle matrix particle compositions further comprise one or more pharmaceutically acceptable excipients as described herein. In certain embodiments, the pharmaceutically acceptable excipient is selected from the group consisting of: a sugar or sugar derivative (e.g., lactose, fructose, mannose, trehalose, amino sugars such as glucosamide or sugar alcohols such as mannitol), an amino acid (e.g., glycine or alanine), and a combination thereof. When the composition comprises an excipient, the excipient may be present from about no excipient to a molar ratio of about 1:9 active pharmaceutical ingredients to the excipient. In some embodiments, the molar ratio of active pharmaceutical ingredients to excipients is from about a composition comprising no excipient to a molar ratio comprising about 1:1 ratio of active pharmaceutical ingredients to excipients. In certain embodiments, the molar ratio of active pharmaceutical ingredients to excipients is about 1:1.

In certain embodiments, a method for making brittle matrix particle compositions described herein comprises: (A admixing a niclosamide compound (e.g., niclosamide) into a solvent wherein the solvent comprises an organic solvent and water to form a pharmaceutical composition wherein the pharmaceutical composition comprises an amount of the niclosamide compound (e.g., niclosamide) in the solvent from about 0.01% to about 10% (NW); (B) applying the pharmaceutical composition to a rotating surface wherein the surface is at a temperature from about −70° C. to about −120° C.; and (C) freezing the pharmaceutical composition to form a brittle matrix pharmaceutical composition. In certain embodiments, the pharmaceutical composition further comprises one or more excipients (e.g., excipients that improve the flow, bioavailability, or control/delay release of the niclosamide compound). Non-limiting examples include: Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl polyvinyl pyrrolidones and tyloxapol, cellulose derivatives, and polyethoxylated castor oil derivatives. In certain embodiments, the amount of the niclsoamide compound in the solvent ranges from about 0.01% (w/v) to about 6% (w/v) (e.g., about 0.1% (w/v) to about 5% (w/v)). In certain embodiments, the method further comprises lyphilizing the brittle matrix, pharmaceutical composition.

The niclosamide compounds described herein (e.g., niclosamide) can be formulated in the brittle matrix particle compositions in an amrophous form or in a particular crystalline form (e.g., in amorphous form). In some embodiments, the brittle matrix particles are low density particles.

The brittle matrix particle compositions can be administered intransally or via respiratory administration (e.g., to the lungs) (e.g., by via aerosols, or via inhalation e.g., through the mouth). The brittle matrix particle compositions may be formulated for use in an inhaler described anywhere herein. Non-limiting examples include metered dose inhaler, a dry powder inhaler, a single dose inhaler, a multi-unit dose inhaler, a nebulizer, or a pressurized metered dose inhaler. In certain embodiments, the brittle matrix particle compositions may be delivered by inhalation and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and may be used in some embodiments. Transmucosal drug delivery in the form of a polytetrafluoroetheylene support matrix is described in U.S. Pat. No. 5,780,045 (specifically incorporated herein by reference in its entirety). As a non-limiting example, the brittle matrix particle compositions may be delivered in aerosols comprising the brittle matrix particles of the niclosamide compounds (and optionally one or more pharmaceutically acceptable excipients) dispersed in a liquified or pressurized gas propellant. For example, the aerosols can comprise a suspension of niclosamide compounds in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

In some embodiments, the brittle matrix particle composition is free of one or more impurities. In certain embodiments, the composition is substantially free of polyvinylpyrrolidone, polyvinylalcohol, polyacrylate, or polystyrene. In certain embodiments, the composition is essentially free of polyvinylpyrrolidone, polyvinylalcohol, polyacrylate, or polystyrene. In certain embodiments, the composition is essentially free of any polymeric excipients. In certain embodiments, the composition is substantially free of poloxamers, polyethylene glycol, or polypropylene glycol. In certain embodiments, the composition is essentially free of poloxamers, polyethylene glycol, or polypropylene glycol. In certain embodiments, the composition is essentially free of any surfactants. In certain embodiments, the composition is free of other compounds beyond the excipient and the active pharmaceutical composition.

The brittle matrix particle composition can be formulated for use in a dry powder inhaler, in certain embodiments, the brittle matrix particle composition comprises a porous flocculated web composition comprising one or more brittle-matrix particles of niclosamide compounds, wherein a portion of the one or more brittle-matrix particles is templated by the subject and/or device induced shearing energy to form porous particles for deep lung delivery. In certain embodiments, the composition comprises porous, matrix of nano-structured primary particles of niclosamide compounds (e.g., niclosamide), upon pulmonary delivery, the nano-structured matrix of primary particles are fractured to release primary particles or aggregates of said primary particles, both of which are smaller than the matrix of nano-structured particles, the fractured particle being appropriate for deep lung delivery. In certain embodiments, the particles of niclosamide compounds (e.g., niclosamide) exhibit a Carr's index of greater than 20 (e.g., greater than 35). In certain embodiments, the porous particles (e.g., fractured particles) have skeletal densities equal to or less than 0.1 glint, (e.g., equal to or less than 0.05 g/mL, equal to or less than 0.01 g/mL). In certain embodiments, the primary particles or aggregates of said primary particles comprise particles having an aerodynamic diameter of between 2 and 5 microns. In certain embodiments, said matrix of nano-structured primary particles are formed by dissolving the niclosamide compound in a solvent to form a niclosamide compound (e.g., niclosamide)/solvent mixture, rapidly freezing niclosamide compound (e.g., niclosamide)/solvent mixture on a cryogenically cooled surface and removing the solvent from the niclosamide/solvent mixture, The brittle-matrix particles may be loaded into the dry powder inhaler by a variety of methods. They may be compacted into blister packs in the solid state. They may also be loaded as colloidal suspensions in a solvent, where the solvent is a liquid, compressed gas, for example a hydrofluroalkane. The evaporation of the solvent may be used to compact the flocs to raise the final particle density in the dry powder inhaler. In addition, the flocs may be formed directly in a component of the dry powder inhale device by thin film freezing. As described above for PMDis, this approach does not use particles that are pre-formed to design the aerodynamic diameter of the aerosol particle. Instead, the aerodynamic diameter is generated in the airways by the shear forces upon rupture of the flocs. This aerodynamic diameter is not present in the starting flocs. Dry powder inhaler suitable for delivering brittle matrix particle compositions include passive inhalation devices with the ability to produce high shear velocities, such as HANDIHALER®.

As a non-limiting example, a dispersible brittle templated composition for a dry powder inhaler system can be prepared by cooling a unit-dose delivery system intended for one or more metered doses for inhalation; depositing one or more drops of a niclosamide compound (e.g., niclosamide) solution on the unit-dose delivery system, wherein the niclosamide solution comprises niclosamide compound (e.g., niclosamide), one or more solvents, and one or more excipients, where said drop freezes upon contact with the packaging material; lyophilizing the pharmaceutical product to produce a non-tightly packed brittle matrix; equilibrating the non-tightly packed brittle matrix to room temperature; and combining the non-tightly packed brittle matrix with a suitable dry powder inhalation device.

In some embodiments, the brittle matrix particle composition can be administered (e.g., via a dry powder inhaler) using a unit-dose delivery system comprising:

one or more concave indentations;

a cover positioned to sealed the one or more concave indentations; and

a composition comprising a niclosamide compound appropriate for pulmonary delivery in at least one of the one or more concave indentations, Wherein the composition comprises porous, matrix of nanostructured primary particles of the niclosamide compound (e.g., niclosamide), wherein said matrix of nano-structured primary particles are formed by dissolving the niclosamide compound (e.g., niclosamide) in a solvent to form a niclosamide/solvent mixture, rapidly freezing niclosamide/solvent on a cryogenically cooled surface and removing the solvent from the niclosamide compound (e.g., niclosamide)/′solvent mixture, further wherein, upon pulmonary delivery, the nano-structured matrix of primary particles are fractured to release primary particles or aggregates of said primary particles, both of which are smaller than the matrix of nano-structured particles, the fractured particle being appropriate for deep lung delivery.

In certain embodiments, the particles of the niclosamide compound (e.g., niclosamide) exhibits a Carr's index of greater than 20 (e.g., greater than 35). In certain embodiments, the porous particles (e.g., the fractured particles) have skeletal densities equal to or less than 0.1 g/mL (e.g., equal to or less than 0.05 g/mL). In certain embodiments, the primary particles or aggregates of said primary particles comprise particles having an aerodynamic diameter of between 2 and 5 microns.

The compositions (e.g., brittle matrix particle compositions) described herein can be formulated in a composition for use in a pressurized metered dose inhaler. A non-limiting exemplary composition comprises a space filled flocculated suspension comprising one or more flocculated particles of the niclosamide compounds (e.g., niclosamide) and a propellant, wherein a portion of the one or more flocculated particles is templated by the formation of one or more droplets upon atomization and whereby the templated floc compacts upon the evaporation of the propellant to form a porous particle for deep lung delivery. In certain embodiments, the one or more flocculated particles comprise one or more anisotropic particles with aspect ratios greater than 1. In certain embodiments, the one or more flocculated particles comprise one or more anisotropic particles with an aspect ratio of about 0.02, 0.05, 0.1, 0.2, 0.3, 0.4, 0.5, 0.6, 0.7, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 5, 10 20, and 50. In certain embodiments, the one or more flocculated particles comprise particles in the form of rods or plates. In certain embodiments, the propellant is a hydrofluoroalkane propellant, such as HFA 134a and HFA 227. In certain embodiments, the one or more flocculated particles are formed by thin film freezing. The composition can be atomized to form an aerosol composition comprising porous particles having one or more at least partially compacted templated flocculated active agents for deep lung delivery. The composition can be prepared using a method comprising: forming a space filled flocculated suspension comprising one or more flocculated particles of one or more active agents and a propellant; templating at least a portion of the one or more flocculated particles by the formation of droplets upon atomization; and compacting the templated floe by the evaporation of the propellant to form a porous particle for deep lung delivery. In certain embodiments, the one or more flocculated particles of anisotropic particles are formed by thin film freezing. See US 2009/0208582 which is incorporated herein by reference in its entirety.

The niclosamide compounds described herein (e.g., niclosamide) can be formulated and delivered via inhalations using one or more formulations, compositions, methods of preparation, and methods of use as described in U.S. patent Ser. No. 10/092,512; U.S. patent Ser. No. 10/434,062; US 2009/0208582; US 2020/0069572; WO 2009/103035; U.S. patent Ser. No. 10/285,945; US 2019/0274958; U.S. Pat. Nos. 8,968,786; 9,622,974; WO 2009/002874; U.S. patent Ser. No. 10/231,955; US 2017/0165238; US 2019/0269661; U.S. Pat. No. 9,044,391; WO 2008/127746; U.S. Pat. Nos. 9,061,027; 9,724,344; WO 2006/026502; US 2004/0022861; U.S. Pat. No. 6,862,890; WO 2002/060411; US 2018/0147161; WO 2016/178704, each of which is incorporated herein by reference in its entirety.

Oral Delivery

In other embodiments, the chemical entities described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Liquid dosage forms for oral administration include pharmaceutically acceptable emulsions, microemulsions, solutions, suspensions, syrups and elixirs. In addition to the chemical entities described herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments, the liquid dosage form is a mouthwash. In certain embodiments, such liquid oral dosage forms are useful for local and topical administration to the digestive or GI tract, e.g., digestive tract, e.g., oral cavity.

Orally administered niclosamide can be delivered to the digestive tract (e.g., the colon) using one or more delivery systems. Non-limiting examples of delivery systems include: prodrugs (e.g., azo-conjugates, pectin prodrugs, or prodrugs formed by conjugation (e.g., through azo-bond) to one or more carrier molecules such as cyclodextrin, glucuronide, dextran, amino acids (e.g., sodium alginate), and HMPC); biodegradable delivery systems (e.g., colon-specific biodegradable delivery systems) (e.g., biodegradable delivery systems using guar gum and derivatives thereof (e.g., AcGGM), azo-aromatic polymers); matrix-based systems (e.g., by embedding niclosamide in polymer matrices such as starch derived polymer matrices (e.g., pH-sensitive and/or biodegradable matrices such as Assam Bora rice starch matrices)); time-released systems (e.g., using pH sensitive polymers); bioadhesive systems (e.g., using polymers such as polycarbophils, polyurethanes, polyethylene oxide, Assam Bora rice starch); multiparticulate systems (e.g., using microspheres (e.g., biodegradable microspheres) such as chitosan microspheres (e.g., coated with Eudragit), guar gum base microspheres, polysaccharide pectins, pectin-4-aminothiophenol (Pec-ATP) conjugates, calcium alginate-carboxymethyl cellulose (CA-CMC), nanoparticles (e.g., with MMT-K10 clay), each of which can be optionally coated with a pH sensitive polymer (e.g., Eudragit)); polysaccharide-based delivery systems (e.g., with xanthan gum, guar gum, pectin (e.g., mixture with an insoluble polymer such as ethyl cellulose), chitosan, HPMC derivatives, chondroitin sulfate, galactomannan, amylose, or combinations of polysaccharides such as combinations of cellulose derivatives (e.g., combinations of non-enteric cellulose esters such as cellulose acetate and enteric cellulose esters such as CAP and HPMCP; e.g., pectin-HPMC, chitosan-HPMC, chitosan-pectin, guar gum-chitosan, and dextran-chitosan)); coatings (e.g., with pH sensitive polymers such as enteric-soluble polymers such as methacrylic-acid based polymers (e.g., Eudragit, Eudragit L, or Eudragit S) or Landolphia owariensis latex (LOL), with acid-soluble polymers such as Eudragit E, with pulsatile coatings, with rupturable film coatings, with permeable or semi-permeable film coatings, and optionally using compression-coating systems wherein the core tablet comprising niclosamide and one or more polymer coatings is further coated with a coating excipient (e.g., almond-gum matrix); pressure-controlled delivery systems; osmotic controlled delivery systems (e.g., OROS-CT); and pulsincap systems. Additional examples are described in Amido, AAPS PharmSciTech, Vol. 16, No. 4, August 2015, which is incorporated herein by reference in its entirety.

Non-limiting examples of oral dosage forms suitable for the selective delivery to the digestive tract (e.g., to the colon) include: delayed release tablets; timed release capsules; immediate release tablets; immediate release capsules (e.g., soft gelatin immediate release capsules); multi-matrix tablets; extended release tablets; gastro-resistant prolonged-release tablets; oral colon-targeted pellets; oral solutions; and oral powders. Additional examples are described in Amido, AAPS PharmSciTech, Vol. 16, No. 4, August 2015, which is incorporated herein by reference in its entirety.

Further non-limiting examples of dosage forms suitable for selective delivery to the digestive tract (e.g., to the colon) include those described in U.S. Pat. Nos. 9,192,583; 6,224,910; 5,914,132; 9,237,760; 9,023,368; 6,228,396; 10,588,864; Int. J. Appl. Res. Nat. Prod., 2012, 5, 1-16; Carbohydrate Polymers, 2013, 92, 367-373; and J. Controlled Release, 1996, 38, 75-94, each of which is incorporated herein by reference in its entirety.

The niclosamide compounds as described herein can be formulated in pharmaceutical compositions disclosed in WO 2019/051437, which is incorporated herein by reference in its entirety. In some embodiments, the pharmaceutical composition comprises: (A) a niclosamide compound (e.g. niclosamide); (B) a pharmaceutically acceptable polymer; and (C) a spontaneously emulsifying component, wherein the emulsifying component comprises: (i) a lipid, solvent, or oil; and (ii) at least 1% w/w relative to the weight of the composition of a surfactant or hydrophilic solvent. The composition can be administered e.g., orally to a subject in need thereof. Methods of making the compositions are described in WO 2019/051437.

In certain embodiments, the pharmaceutically acceptable polymer is a cellulosic polymer, such as a neutral cellulosic polymer or an ionizable cellulosic polymer, or a non-cellulosic polymer (e.g., a neutral non-cellulosic polymer or an ionizable non-cellulosic polymer). In certain embodiments, the pharmaceutically acceptable polymer is a polymethacrylate or polyacrylate functionalized with a carboxylic acid group. In certain embodiments, one or more of lipids, solvent, or oils is in the liquid phase. In certain embodiments, the emulsifying composition comprises a lipid or oil. In certain embodiments, the lipid or oil is an ester of a fatty acid. In certain embodiments, the lipid or oil is a glyceride ester of one, two, or three fatty acids. In certain embodiments, the fatty acids is medium chain fatty acids. In certain embodiments, the lipid or oil is Capmul®.

In certain embodiments, the spontaneously emulsifying composition comprises a solvent, such as a hydrophobic solvent (e.g., a solvent that contains one or more aromatic groups such as benzyl benzoate). In certain embodiments, the spontaneously emulsifying composition comprises a surfactant or hydrophilic solvent that contains one or more polyethylene glycol or polypropylene glycol repeating units. In certain embodiments, the hydrophilic solvent is a PEG polymer, such as a PEG polymer with a molecular weight from 100 Daltons to 2000 Daltons (e.g., PEG 200 or PEG 400).

In certain embodiments, the pharmaceutical composition comprises a first surfactant. In certain embodiments, first surfactant is polyethoxylated castor oil such as Cremophor EL. In certain embodiments, the pharmaceutical composition further comprises a second surfactant. In certain embodiments, the second surfactant is a compound with a hydrophobic component and a PEG or polypropylene glycol component. In certain embodiments, the hydrophobic component is a fatty acid. In certain embodiments, the PEG or polypropylene glycol component is a PEGylated polysorbate. In certain embodiments, the second surfactant is Tween®.

In certain embodiments, the niclosamide compound (e.g., niclosamide) comprises from about 10% w/w to about 60% w/w of the total weight of composition (e.g., from about 20% w/w to about 50% w/w, e.g., from about 20% w/w to about 40% w/w). In certain embodiments, the niclosamide compound (e.g., niclosamide) is present at a concentration greater than the solubility of the niclosamide compound (e.g., niclosamide) in the pharmaceutically acceptable polymer alone. In certain embodiments, the niclosamide compound (e.g., niclosamide) is present at a concentration greater than the solubility of the niclosamide compound (e.g., niclosamide) in the spontaneously emulsifying component alone. In certain embodiments, the niclosamide compound (e.g., niclosamide) is present at a concentration greater than the solubility of the niclosamide compound (e.g., niclosamide) in either the pharmaceutically acceptable polymer or the spontaneously emulsifying component alone. In certain embodiments, the niclosamide compound (e.g., niclosamide) is present at a concentration greater than the solubility of the niclosamide compound (e.g., niclosamide) in the pharmaceutically acceptable polymer or the spontaneously emulsifying component combined. In certain embodiments, the niclosamide compound (e.g., niclosamide) is formulated such that at least 10% of the niclosamide compound (e.g., niclosamide) is present in the undissolved form when added to or diluted in physiological fluid or water. For example, at least 50% of the niclosamide compound (e.g., niclosamide) is present in the undissolved form when added to or diluted in physiological fluid or water.

In certain embodiments, the pharmaceutically acceptable polymer comprises from about 20% w/w to about 80% w/w of the total weight of the composition. In certain embodiments, the pharmaceutically acceptable polymer comprises from about 40% w/w to about 80% w/w (e.g., from about 50% w/w to about 80% w/w).

In certain embodiments, the lipid, oil, or solvent comprises from about 0.25% w/w to about 10% w/w of the total weight of composition (e.g., from about 0.5% w/w to about 5% w/w, e.g., from about 1% w/w to about 3% w/w). In certain embodiments, the surfactant comprises from 2% w/w to about 10% w/w of the total weight of composition (e.g., from 2% w/w to about 6% w/w, e.g., from 3% w/w to about 5% w/w). In certain embodiments, the hydrophilic solvent comprises from 2% w/w to about 10% w/w of the total weight of composition (e.g., from 2% w/w to about 6% w/w, e.g., from 3% w/w to about 5% w/w). In certain embodiments, the second surfactant comprises from 2% w/w to about 10% w/w of the total weight of composition (e.g., from 2% w/w to about 6% w/w, e.g., from 3% w/w to about 5% w/w).

The niclosamide compounds as described herein can be formulated in pharmaceutical compositions disclosed in WO 2019/051440, which is incorporated herein by reference in its entirety. In some embodiments, the pharmaceutical composition comprises: (A) a niclosamide compound e.g., niclosamide), wherein the niclosamide compound (e.g., niclosamide) comprises at least about 50% w/w of the pharmaceutical composition; (B) one or more pharmaceutically acceptable polymers; and (C) a non-preloaded mesoporous carrier. The composition can be administered e.g., orally to a subject in need thereof. Methods of making the compositions are described in WO 2019/051440.

In certain embodiments, the pharmaceutically acceptable polymer is a cellulosic polymer, such as a neutral cellulosic polymer or an ionizable cellulosic polymer. In certain embodiments, the pharmaceutically acceptable polymer is a non-cellulosic polymer, such as a neutral non-cellulosic polymer or an ionizable non-cellulosic polymer. In certain embodiments, the pharmaceutically acceptable polymer is an ionizable polymethacrylate or polyacrylate. In certain embodiments, the pharmaceutically acceptable polymer is a polymethacrylate or polyacrylate functionalized with a carboxylic acid group.

In certain embodiments, the mesoporous carrier is a silica carrier, an alumina carrier, a mixed alumino-silicate carrier, a mixed inorganic oxide carrier, a calcium carbonate carrier, or a clay carrier, such as a mesoporous silica or silicate (e.g., a mesoporous silica). As a non-limiting example, the mesoporous carrier is a hydrous silicon dioxide (e.g., mesoporous fumed silica, such as a mesoporous magnesium aluminum silicate). In certain embodiments, the mesoporous carrier has not been loaded with the niclosamide compound before the formulation with the pharmaceutically acceptable polymer. In certain embodiments, the mesoporous carrier has not been loaded with any therapeutic agent prior to formulation with the niclosamide compound and the pharmaceutically acceptable polymer.

In certain embodiments, the pharmaceutically acceptable polymer and the niclosamide compound (e.g., niclosamide) form a mixture having a Flory-Huggins interaction parameter (x) of greater than 0.25 as determined by differential scanning calorimetry (DSC). In certain embodiments, the Flory-Huggins interaction parameter (χ) is greater than 1. In certain embodiments, the pharmaceutically acceptable polymer and the niclosamide compound form a mixture having a positive AGmix as determined by DSC.

In certain embodiments, the pharmaceutical composition has a specific surface area of greater than about 5 m²/g as measured by BET (e.g., greater than about 10 m²/g, greater than about 15 m²/g, or greater than about 20 m²/g).

In certain embodiments, the pharmaceutical composition comprises from about 50% w/w to about 98% w/w niclosamide compound (e.g., niclosamide) relative to the total weight of the pharmaceutical composition (e.g., from about 50% w/w to about 75% w/w, e.g., from about 50% w/w to about 60% w/w). In certain embodiments, the pharmaceutical composition comprises from about 1% w/w to about 49% w/w mesoporous carrier relative to the total weight of the pharmaceutical composition. In certain embodiments, the pharmaceutical composition comprises from about 10% w/w to about 40% w/w mesoporous carrier relative to the total weight of the pharmaceutical composition. In certain embodiments, the pharmaceutical composition comprises from about 15% w/w to about 35% w/w mesoporous carrier relative to the total weight of the pharmaceutical composition (e.g., 25% w/w to about 35% w/w). In certain embodiments, the pharmaceutical composition comprises from about 1% w/w to about 49% w/w pharmaceutically acceptable polymer relative to the total weight of the pharmaceutical composition (e.g., from about 10% w/w to about 40% w/w, e.g., from about 15% w/w to about 35% w/w, e.g., from about 15% to about 25%).

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phosphate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a niclosamide compound, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the chemical entity is a niclosamide compound, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a niclosamide compound, or a pharmaceutically acceptable salt and/or hydrate and/or cocrystal thereof) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a niclosamide compound, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) is an oral solid dosage form and includes the ingredients and amounts as shown in Table 7.

TABLE 7

| Ingredient | Weight Percent |
|---|---|
| niclosamide compound | 40 weight percent to about 80 weight |
| | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone | 0.5 weight percent to about 5 weight |
| | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| | percent (e.g., from about 20 weight percent |
| | to about 40 weight percent, from about 25 |
| | weight percent to about 35 weight percent; |
| | e.g., about 31.03 weight percent |
| Povidone | about 0.5 weight percent to about 10 weight |
| | percent (e.g., from about 2 weight |
| | percent to about 8 weight percent, from |
| | about 3 weight percent to about 6 weight |
| | percent; e.g., about 5 weight percent |
| Magnesium stearate | about 0.05 weight percent to about 3 |
| | weight percent (e.g., from about 0.05 |
| | weight percent to about 2 weight percent; |
| | from about 0.1 weight percent to about 1.5 |
| | weight percent; e.g., about 1 weight percent |

In certain embodiments, component (1) includes the ingredients and amounts as shown in Table 8.

TABLE 8

| Ingredient | Weight Percent |
|---|---|
| niclosamide compound | About 66.7 weight percent |
| Crospovidone | About 5.00 weight percent |
| lactose monohydrate | About 24.63 weight percent |
| Povidone | About 2.70 weight percent |
| Magnesium stearate | About 1.00 weight percent |

In certain embodiments, component (1) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging. See, e.g., Example 8.

In certain of the foregoing embodiments, component (ii) includes water and one or more (e.g., all) of the following excipients (e.g., as a liquid phase for an enema):
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phosphate dihydrate, disodium phosphate dodecahydrate);

n certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′) is present.

In certain embodiments, component (ii) includes water (up to 100%) and the ingredients and amounts as shown in Table 9.

TABLE 9

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 100%) and the ingredients and amounts as shown in Table 10.

TABLE 10

| Ingredient | Weight Percent |
|---|---|
| methyl cellulose | about 1.4 weight percent |
| (Methocel A15C premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 1A-1C, which includes a plastic bottle, a breakable capsule, and a rectal cannula and single flow pack.

Ocular Delivery

In some embodiments, niclosamide, or a pharmaceutically acceptable salt thereof, or a pharmaceutical composition thereof is suitable for local and topical administration to the eye (e.g., eye drops, ocular ointments, ocular gels, contact lenses, and ophthalmic inserts). See, e.g., Dubald et al. Pharmaceutics. 2018; 10(1): 10 and Patel et al. World J Pharmacol. 2013; 2(2): 47-64. In some embodiments, compositions suitable for ocular delivery include in situ gelling systems, liposomes, nanoparticles, niosomes, nanoemulsions, and microemulsions Ocular compositions can include, without limitation, one or more of any of the following: viscogens (e.g., polyvinylalcohol (PVA), hydroxylmethylcellulose, hydroxylethylcellulose carboxymethylcellulose, glycerin, polyvinylpyrrolidone, polyethylene glycol); stabilizers (e.g., pluronic (triblock copolymers), cyclodextrins); preservatives (e.g., benzalkonium chloride, ETDA, SofZia (boric acid, propylene glycol, sorbitol, and zinc chloride; Alcon Laboratories, Inc.), purite (stabilized oxychloro complex; Allergan, Inc.)); permeation enhancers (e.g., polyoxyethylene glycol ester and ethylenediaminetetra acetic acid sodium salt); and lubricants. In some embodiments, a composition for ocular delivery is isotonic.

In some embodiments, an ocular ointment includes non-aqueous excipients. In some embodiments, an ocular ointment has an oleaginous base, an absorption base, a water-removable base, or a water soluble base. An oleaginous base can be a lipophilic ointment. For example, an oleaginous base can include petrolatum and white ointment. An adsorption base can be used as emollient. For example, an adsorption base can include lanolin, fatty alcohol and petrolatum. A water-soluble base can include only water soluble excipients such as macrogol with high molecular weight. A water removable base includes compositions that are an oil in water emulsion.

In some embodiments, an ocular gel is a hydrogel. For example, a preformed gel or a composition that forms a gel in situ. Hydrogels can include polymers such as methylcellulose, hydroxylethylcellulose, sodium hyaluronate, sodium alginate, povidone, polyvinylalcohol, cellulose acetate and derivatives, carbomer, magrogol, pseudolatex, polymethacrylic acid, alginate sodium, gellan gum (GELRITE®), pluronics, poly(n-isopropyl acrylamide), oly(acrylic acid), polyacrylamide, poloxamer, chitosan, and hydroxyl propyl methyl cellulose.

In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Other Forms of Delivery

In some embodiments, the chemical entities described herein, or a pharmaceutical composition thereof, are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In some embodiments, the chemical entities described herein, or a pharmaceutical composition thereof, are suitable for administration to endothelial cells. For example, niclosamide, or a pharmaceutically acceptable salt thereof, can be formulated for intravenous or oral administration.

In some embodiments, niclosamide, or a pharmaceutical composition thereof, is suitable for local administration to endothelial cells. For example, niclosamide, or a pharmaceutically acceptable salt thereof, can be locally delivered to endothelial cells using a stent (e.g., a drug-eluting stent). See, e.g., Finkelstein et al. Circulation. 2003; 107:777-784; Tzafriri et al. Intery Cardiol Clin. 2016 July; 5(3):307-320; and Yoon et al. Neurosurg Focus. 2017 April; 42(4):E11. In some embodiments, niclosamide, or a pharmaceutically acceptable salt thereof, is formulated for targeted delivery to endothelial cells. In some embodiments, compositions comprising niclosamide suitable for targeted delivery to endothelial cells include liposomes (e.g., liposomal drug delivery vehicles), polymersomes, and nanoparticles (e.g., solid PEG-copolymer nanoparticles). Non-limiting examples of liposomes suitable for targeted delivery of niclosamide to endothelial cells include PEG-coated liposomal drug vehicles.

In some embodiments, the liposome comprises phospholipids. In some embodiments, the polymersome comprises amphiphilic diblock copolymers such as degradable PEG-poly(caprolactone). In some embodiments, niclosamide, or a pharmaceutically acceptable salt thereof, is conjugated to PEG.

In some embodiments, the composition comprises a ligand that binds to an endothelial cell surface marker. For example, the ligand can be conjugated to a liposome, polymersome, or nanoparticle comprising niclosamide. In some embodiments, the endothelial cell surface marker comprises constitutive angiotensin-converting enzyme (ACE), thrombomodulin, a caveolar protein such as aminopeptidase P2, a growth factor, an integrin, a transferrin receptor, a cell adhesion molecule (CAM), or a combination thereof. Non-limiting examples of CAMs include P- and E-selectins, Ig-family members such as Platelet-Endothelial Cell Adhesion Molecule-1 (PECAM-1), Intercellular Adhesion Molecule-1 (ICAM-1), and Vascular Cell Adhesion Molecule 1 (VCAM-1). In some embodiments, the ligand comprises an antibody or a portion of an antibody against an endothelial cell surface marker. In some embodiments, the ligand is a scFv/TM fusion protein or a scFv/endothelial protein C receptor (EPCR) fusion protein. In some embodiments, the ligand is a PECAM-1 specific scFv/TM fusion protein, an ICAM-1 targeted scFv-thrombomodulin fusion protein, a Mec13 scFv/EPCR fusion protein, or a combination thereof. See, e.g., Simone et al. Cell Tissue Res. 2009 January; 335(1): 283-300 and Kiseleva et al. Drug Deliv Transl Res. 2018; 8(4): 883-902.

In some embodiments, a composition formulated for targeted delivery to endothelial cells is administered intravenously to a subject.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, a niclosamide compound is administered is administered at a dosage of from about 0.01 mg/Kg to about 200 mg/Kg (e.g., from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg).

In certain embodiments, the niclosamide compound is administered at a dosage of from about 15 mg/Kg to about 100 mg/Kg (e.g., from about 15 mg/Kg to about 90 mg/Kg, from about 20 mg/Kg to about 100 mg/Kg; from about 20 mg/Kg to about 90 mg/Kg; from about 20 mg/Kg to about 80 mg/Kg; from about 30 mg/Kg to about 90 mg/Kg; from about 30 mg/Kg to about 80 mg/Kg; from about 35 mg/Kg to about 75 mg/Kg; from about 10 mg/Kg to about 50 mg/Kg; from about 15 mg/Kg to about 45 mg/Kg; e.g., about 35 mg/Kg or about 75 mg/Kg). In other embodiments, the chemical entity is administered at a dosage of from about 0.1 mg/Kg to about 10 mg/Kg (e.g., from about 0.1 mg/Kg to about 5 mg/Kg; from about 1 mg/Kg to about 10 mg/Kg; from about 1 mg/Kg to about 5 mg/Kg).

In some embodiments, formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the niclosamide compound. In certain embodiments, said dosages are suitable for formulations administered by rectal administration (e.g., by enema).

In certain embodiments, formulations include from about 50 mg to about 250 mg (e.g., from about 100 mg to about 200; e.g., about 150 mg) of the niclosamide compound. In certain embodiments, said dosages are suitable for formulations administered by rectal administration (e.g., by enema).

In some embodiments, formulations include from about 500 mg to about 2500 mg (e.g., from about 600 mg to about 1800 mg, from about 700 mg to about 1700 mg, from about 800 mg to about 1600 mg, from about 900 mg to about 1500 mg, from about 1000 mg to about 1400 mg, from about 1100 mg to about 1300 mg, e.g., about 1200 mg. In certain embodiments, said dosages are suitable for formulations administered by oral administration (e.g., by tablet or pill).

In certain embodiments, formulations include from about 100 mg to about 700 mg (e.g., from about 200 mg to about 600 mg; e.g., from about 300 mg to about 500 mg; e.g., from about 350 mg to about 450 mg; e.g., about 400 mg) of the niclosamide compound. In certain embodiments, said dosages are suitable for formulations administered by oral administration (e.g., by tablet or pill).

The foregoing dosages can be administered on a daily basis (e.g., as a single dose per day; or as two or more divided doses per day; or a two or more doses; e.g., two doses per day; or three doses per day; or four doses per day; or five doses per day; e.g., three doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond; e.g., 2 weeks.

For example, dosages (e.g., about 2.5 mg/mL or about 7.5 mg/mL) of the chemical entity in liquid carrier can be administered twice a day on a daily basis for about 6 weeks. In certain of these embodiments, the chemical entity is niclosamide, or a pharmaceutically acceptable salt and/or hydrate and/or cocrystal thereof. For example, about 2.5 mg/mL or about 7.5 mg/mL of niclosamide in liquid carrier can be administered twice a day on a daily basis for about 6 weeks. Representative liquid carriers include, e.g., those previously described in conjunction with component (ii).

In certain embodiments, formulations include from about 100 mg to about 700 mg (e.g., from about 200 mg to about 600 mg; e.g., from about 300 mg to about 500 mg; e.g., from about 350 mg to about 450 mg; e.g., about 400 mg) of the niclosamide compound, and the foregoing dosages are administered on a daily basis. In certain embodiments, the foregoing dosages are administered as a single dose per day (e.g., for 14 days). In certain embodiments, said dosages are suitable for formulations administered by oral administration (e.g., by tablet or pill).

In some embodiments, formulations include from about 500 mg to about 2500 mg (e.g., from about 600 mg to about 1800 mg, from about 700 mg to about 1700 mg, from about 800 mg to about 1600 mg, from about 900 mg to about 1500 mg, from about 1000 mg to about 1400 mg, from about 1100 mg to about 1300 mg, e.g., about 1200 mg. In certain embodiments, the foregoing dosages are administered on a daily basis. In certain embodiments, the foregoing dosages are administered as two or more divided doses per day; or a two or more doses; e.g., two doses per day; or three doses per day; or four doses per day; or five doses per day; e.g., three doses per day); e.g., for 14 days. In certain embodiments, said dosages are suitable for formulations administered by oral administration (e.g., by tablet or pill). In certain embodiments, the foregoing dosages are administered as two or more divided dosages per day, e.g., three doses per day; e.g., three 400 mg dosages per day; e.g., for 14 days.

### PROPHETIC EXAMPLES

### Example 1: A Randomized Double Blind, Placebo-Controlled Study on the Safety and Efficacy of Niclosamide in Patients with Long Hauler's Syndrome Associated with Covid-19

### Overview

### Study Design

This is a multicentre, randomized, double blind, 2 arm placebo-controlled study in adults with long hauler's syndrome associated with COVID-19. The study will enroll outpatients with two study groups with stratified and randomized enrollment.

### Purpose

The purpose of this study is to evaluate the safety and efficacy of niclosamide in addition to current standard of care (SoC) compared to placebo in patients presenting with long hauler's syndrome associated with COVID-19.

### Number of Subjects

The study will include approximately 100 patients who are not planned to be hospitalized with an option to increase to approximately 130.

### Patient Sopulation

The study population will include approximately 100 to 130 patients who are not planned to be hospitalized.

In both parts, eligible patients will have been diagnosed with long hauler's syndrome associated with COVID-19. Each patient should meet all the inclusion and none of the exclusion criteria in order to be eligible for the study.

### Objectives

The objectives of this study are to evaluate the clinical efficacy, safety, and tolerability of oral niclosamide in addition to SOC compared to placebo in addition to SOC.

### Endpoints

Gastrointestinal efficacy secondary endpoints are: a) time from the first dose of niclosamide to the first formed stool (this formed stool must have been followed by a non-watery stool); b) time from the first dose of niclosamide to the last watery stool;

Systemic and respiratory efficacy secondary endpoints are: d) clinical severity score (as recommended by the World Health Organization for COVID-19 studies) over time; e) duration, type of administration and quantity of supplemental oxygen treatment; f) changes in body temperature; g) blood oxygen saturation on room air (by pulse oximetry) over time; h) portion of patients requiring ICU admission and length of ICU stay j) portion of patients requiring hospitalization and duration of hospitalization.

Safety and tolerability endpoints are: k) all-cause mortality 6 weeks after randomization number of patients with 1) portion of patients with Treatment Emergent Adverse Events (TEAE) leading to study drug discontinuation; m) serious adverse event (SAE), n) treatment-emergent adverse event (TEAE), clinically significant changes in o) laboratory measures, and p) vital signs.

### Dosage

Group 1 (active): Continued SOC therapy together with niclosamide 400 mg tablets TID (total daily dose 1,200 mg) for 14 days.

Group 2 (placebo): Continued SOC therapy together with placebo tablets matching niclosamide TID for 14 days.

### Randomization and Stratification

Patients will be randomized 1:1 between Group 1 (active) and Group 2 (placebo) following stratification based on age, use of concomitant antiviral therapies, and female or male sex.

### Visit Schedule

Screening and informed consent will occur rapidly (up to 3 Days before Day 1). Dosing will initiate after confirmation of eligibility and randomization on Day 1.

Dosing will continue for 14 days. If the patient is hospitalized, efforts will be made to continue therapy and assessments while hospitalized; hospital discharge is at the discretion of the medical staff at the hospital. Study assessments will continue through Day 43. The final evaluation for safety and efficacy is planned to occur on Day 43.

### Sample Size Calculation

The sample size will be determined by simulation. As described below, with a one-sided test for shorter time to clearance with niclosamide, a type-I error rate of 5%, and 50 patients per arm (100 patients total completing the study with an evaluable primary endpoint) the study is estimated to achieve approximately 96% power. In some scenarios 96% may appear to be over-powered, but, given the exploratory nature of the study and the unknown true effect size, the number of patients appears reasonable.

### Statistical Methods

The primary analysis of the study will be a stratified log-rank test. Stratification will be based on strata defined for randomization. Kaplan-Meier curve figures with 90% confidence intervals will be generated for the total population by treatment and for each stratum by treatment. Tables will present summary statistics of fraction of subjects not cleared by planned visit, and these summary tables will illustrate the total population and each stratum by treatment. Listings of all patients and the time to the primary endpoint along with stratum levels and clarifications required for sensitivity analyses will be generated.

Secondary and exploratory endpoints along with safety and baseline characteristics will be summarized with tables, figures, and listings appropriate for each measure.

TABLE 11
List of Abbreviations

| ABBREVIATIONS | DEFINITIONS |
|---|---|
| ACE2 | Angiotensin Converting Enzyme 2 |
| ADR | Adverse Drug Reaction |
| AE | Adverse Event |
| ALT | Alamine amino-Transferase |
| AST | Aspartate amino-Transferase |
| ATC | Anatomical Therapeutic Chemical coding |
| BP | Blood pressure |
| CI | Confidence Interval |
| CPM | Copies Per Milliliter |
| CRA | Clinical Research Associate |
| CRO | Contract Research Organization |
| DBP | Diastolic Blood Pressure |
| DDD | Drug Daily Dose |
| DM | Data Manager |
| DMC | Data Monitoring Committee |
| DMP | Data Management Plan |
| e-CRF | Electronic Case Report Form |
| FAS | Full Analysis Set |
| FPFV | First Patient First Visit |
| GCP | Good Clinical Practice |
| GGT | Gamma-Glutamyl Transferase |
| GI | Gastrointestinal |
| GMP | Good Manufacturing Practice |
| Hr | Hour |
| ICF | Informed Consent Form |
| ICU | Intensive Care Unit |
| IEC | Institutional Ethic Committee |
| ITT | Intention To Treat |
| IMP | Investigational Medical Product |
| IWRS | Interactive Web Randomization System |
| LPLV | Last Patient Last Visit |
| MedDRA | Medical Dictionary for Regulatory Activities |
| MERS | Middle East respiratory Syndrome |
| PT | Preferred Term |
| RT-qPCR | Reverse transcriptase-quantitative polymerase |
| | chain reaction |
| SAE | Serious Adverse Event |
| SaO2 | Blood Oxygen Saturation by Pulse Oximetry |
| SAP | Statistical Analysis Plan |
| SARS | Severe Acute Respiratory Syndrome |
| SBP | Systolic Blood Pressure |
| SD | Standard Deviation |
| SOC | Standard of Care |
| SOC | System Organ Class |
| TEAE | Treatment Emergent Adverse Event |
| TID | Three times per day (Ter In Die) |
| ULN | Upper Limit of Normal |
| WHO | World Health Organization |

### Rationale for the Study

There are currently no approved or investigational treatments with demonstrated clinical efficacy for long hauler's syndrome associated with COVID-19. The evaluation of a safe and effective antiviral agent will meet a serious unmet medical and epidemiological needs. Considering the objective of this protocol, the overall risks to participants are outweighed by the potential benefits of niclosamide experimental therapy. For these reasons the benefit-risk balance for this study is considered positive.

### Risk/Benefit Assessment

### Risk for the Patient

A specific risk for a patient participating in this study is the exposure to the adverse reactions of niclosamide. This drug was approved by for use in humans as anti-helminthic in early 1980's and is included in the World Health Organization's list of essential medicines (WHO, “The Selection and Use of Essential Medicines”, World Health Organization, Geneva, 2007, which is hereby incorporated by reference in its entirety).

To date niclosamide has been administered to millions of people, including children, and appears to have a very good safety profile: gastro-intestinal disturbance is occasionally reported, whereas light-headedness and pruritus have been reported less frequently (Martindale: The Complete Drug Reference (37th ed, 2012), which is hereby incorporated by reference in its entirety). Nausea, retching and abdominal pain are also reported (WHO, WHO model formulary, (2008), which is hereby incorporated by reference in its entirety).

Other reported AEs (1-4% of patients) are: vomiting; abdominal discomfort possibly associated with anorexia, diarrhea, drowsiness, dizziness and headache. Less frequently reported (<1%) AEs are: rash, oral irritation, fever, rectal bleeding, weakness, bad taste in mouth, sweating, palpitation, constipation, alopecia, oedema of an arm, backache, and irritability. A transient increase in serum AST has reportedly occurred in at least one patient who was physically dependent on opiate agonists. Urticaria, which may be caused by the presence of breakdown products of the dead or dying worms, has occurred rarely (McEvoy, G. K. “American Hospital Formulary Service-Drug Information 95”, Bethesda, Md.: American Society of Hospital Pharmacists, Inc., 1995 Plus Supplements 1995, which is hereby incorporated by reference in its entirety, p. 44).

### Other Risks

The examinations and procedures required by the study do not expose the investigator or healthcare professionals to risks other than those that would normally involve the management of long hauler's syndrome associated with COVID-19 patients.

### Potential Benefit for the Patient

If the gut-selective antiviral activity of niclosamide were confirmed, the potential therapeutic or prophylactic return for the patient would be.

a. Effects on GI symptoms, and diarrhea in particular, due to the control of the viral replication within the epithelial cells and reduced interaction with ACE2 receptors.

b. Effects on liver complications, likely as a consequence of the spread of the virus through the enterohepatic circulation.

c. Possible effects on respiratory and systemic symptoms due to the reduction of circulating viral load of enteric origin.

d. Potential systemic effects due to, even if limited, intestinal absorption and potential model for the development of formulations acting at systemic level.

e. Niclosamide acts locally at the intestinal level, and due to its poor systemic absorption, it is suitable to be associated with other systemic treatments for long hauler's syndrome associated with COVID-19 without expected pharmacokinetic drug interactions.

For patients with a positive local laboratory test and a negative central laboratory test, the potential benefits relate to the fact that the patient will likely have SARS-CoV-2 infection based on the other inclusion criteria and the fact that the local laboratory test was positive. The potential for a false-negative at the central laboratory following a potential true-positive at the local laboratory would indicate that the patient may receive benefit as with the other patients in the trial.

### Regulatory Compliance

This study will be conducted in compliance with this protocol, Good Clinical Practice (ICH-GCP), all applicable national and local regulatory requirements and in accordance with the ethical principles that have their origin in the Declaration of Helsinki and following revisions.

The investigator is responsible for ensuring the study is conducted in accordance with the procedures described in this protocol.

### Objectives

### Primary Objective

The objectives of this study are to evaluate the clinical efficacy, safety, and tolerability of oral niclosamide in addition to SOC compared to placebo in addition to SOC.

### Exploratory Objective

The exploratory objective of this study is to evaluate biomarkers relating administration of niclosamide or placebo to the drug under study, virologic response, inflammatory response, symptoms or disease severity.

### Endpoints

### Primary Endpoint

The objectives of this study are to evaluate the clinical efficacy, safety, and tolerability of oral niclosamide in addition to SOC compared to placebo in addition to SOC.

### Secondary Objectives and Other Endpoints

### Gastrointestinal Efficacy Secondary Endpoints

a) Time from the first dose of niclosamide to the first formed stool (this formed stool must have been followed by a non-watery stool).

b) Time from the first dose of niclosamide to the last watery stool.

c) Proportion of patients administered any anti-diarrheal agent from the first dose of niclosamide to Day 15 and from Day 16 to 29.

### Systemic and Respiratory Efficacy Secondary Endpoints

d) Proportion of patients with each clinical severity score as recommended by the World Health Organization (WHO) for COVID-19 studies (Table 13) by study visit.

e) Total duration, type of administration (e.g. mean increased room oxygen, nasal tubes, ventilator, and ECMO), and quantity of supplemental oxygen treatment, whenever possible.

f) Body temperature and portion of patients with normal body temperature by study day. Criteria for normalization: temperature: <36.0° C. axillary, <36.6° C. oral, <37.0° C. rectal, <36.6° C. tympanic (Geneva II et al., 2019).

g) Proportion of patients with normal blood oxygen saturation (SaO2, fingertip pulse oximeter), >90%, on room air by study day.

h) Proportion of patients admitted to the intensive care unit (ICU) and length of ICU stay.

i) Time to nasopharyngeal SARS-CoV-2 virus clearance from the nasopharynx (assessed by RT-qPCR) in the niclosamide group, compared to the placebo group.

j) Duration of hospitalization.

k) proportion of patients admitted to hospital by Day 14.

TABLE 13
WHO Ordinal Scale for Clinical Improvement. A special WHO
(2020) committee arrived at the ordinal scale in the table
below that measures illness severity over time.

| Patient State | Descriptor | Score |
|---|---|---|
| Uninfected | No clinical or virologic | 0 |
| | evidence of infection | |
| Ambulatory | No limitation of activities | 1 |
| Ambulatory | Limitation of activities | 2 |
| Hospitalised, | Hospitalised, no oxygen | 3 |
| Mild Disease | therapy | |
| Hospitalised, | Oxygen by mask or nasal | 4 |
| Mild Disease | prongs | |
| Hospitalised, | Non-invasive ventilation | 5 |
| Mild Disease | or high-flow oxygen | |
| Hospitalised, | Intubation and mechanical | 6 |
| Mild Disease | ventilation | |
| Hospitalised, | Ventilation + additional | 7 |
| Mild Disease | organ support - pressors, | |
| | RRT, ECMO | |
| Dead | Dead | 8 |

### Safety and Tolerability Endpoints

l) All-cause mortality 6 weeks after randomization.
m) Proportion of patients with Treatment Emergent Adverse Events (TEAE) leading to study drug discontinuation.

n) Serious adverse event (SAE) coded by System Organ Class (SOC) and Preferred Term (PT), using the Medical Dictionary for Regulatory Activities (MedDRA.

o) Clinically significant changes in laboratory measures.

p) Clinically significant changes in vital signs.

### Exploratory Endpoints

q) All residual samples will be retained, as allowed, to allow for evaluation the drug under study, virologic response, or disease severity.

### Research Method

Patients will be recruited in public hospitals and in outpatient medical settings that may include telemedicine options and will be randomized 1:1 to niclosamide oral formulation plus SOC or placebo matching niclosamide tablets plus SOC.

Study Design

This is a multicentre, randomized, double blind, 2-arm placebo-controlled study in adults with long hauler's syndrome associated with COVID-19.

Study Design

The two study groups will be randomized in a 1:1 ratio using an IWRS and balanced and stratified by age, use of concomitant antiviral therapies allowed in United States for use (including off-label use) in COVID-19, and female or male sex:

1) Age:
a) <65 years old
b) ≥65 years old

3) Sex:
a) Female
b) Male

### Study Treatment and its Duration

Approximately 100 to 130 patients who meet all inclusion and exclusion criteria are planned to be randomized in a 1:1 ratio into one of the following two treatment groups:
Group 1 (active): Continued SOC therapy together with niclosamide 400 mg tablets three times per day (TID), equivalent to a total daily dose 1,200 mg, for 14 days.
Group 2 (placebo): Continued SOC therapy together with placebo tablets matching niclosamide TID for 14 days.

Treatment may begin at any time of day, and if only one or two doses is administered on Day 1, the planned total number of doses should be administered (42 total doses) where the final doses may be administered on Day 16. The total study duration for a patient will not be extended if the final dose occurs on Day 16 for this reason.

The long-term care of the participant will remain the responsibility of their primary treating physician and there is no provision for post-study availability of niclosamide.

### Study Duration and Recruitment Period

Each patient will remain in the study for a total of 6 weeks, with a treatment duration of 14 days. The recruitment period will last approximately 5 months.

The total study duration from First Patient First Visit (FPFV) to Last Patient Last Visit (LPLV) is expected to be 6 months approximately.

### Population

The study population will include 100 evaluable adult patients who have been diagnosed with long hauler's syndrome associated with COVID-19 who are not expected to be hospitalized at the time of randomization.

### Both Parts:

Patients will have positive pre-dose test results for SARS-CoV-2 in a rectal swab or a stool sample by the central laboratory, have diarrhea with or without respiratory symptoms. No gender and/or ethnicity restrictions will apply. Each patient should meet all the inclusion and none of the exclusion criteria in order to be eligible for the study.

### Inclusion Criteria

Subjects meeting all the following inclusion criteria will be considered eligible for the study:

1. Patients who gave their written consent for participation in the study and for personal data processing and are willing to comply with all study procedures.

2. Males or females aged 18 years or more.

3. Patients with a primary diagnosis of long hauler's syndrome associated with COVID-19 with or without pneumonia who are not planned to be hospitalized.

4. Body weight between 45 kg and 145 kg, inclusive, at screening

5. Patients who are reasonably expected to maintain and continue taking, after randomization, their prescription for antiviral drugs.

6. Patients who prior to developing long hauler's syndrome associated with COVID-19 usually have normal bowel habits defined as at least solid-formed 3 stools per week and no more than 3 solid-formed stools per day.

7.

8.

### Exclusion Criteria

Patients meeting any of the following exclusion criteria will not be eligible for the study:

1. Cannot obtain informed consent.

2. Pregnant or lactating women or women with a positive pregnancy test.

3. At the time of randomization patients requiring ICU admission or patients with severe respiratory insufficiency requiring mechanical ventilation or with rapid worsening of respiratory function leading to expectation for mechanical ventilation or ICU admission.

4. Evidence of rapid clinical deterioration or existence of any life-threatening co-morbidity or any other medical condition which, in the opinion of the investigator, makes the patient unsuitable for inclusion.

5. Patients who, at the time of enrolment, are not in a clinical condition compatible with the oral administration of the study drug.

6. Serum alanine transaminase (ALT) or aspartate transaminase (AST) >3 times upper limit of normal detected within 24 hours at screening or at baseline or other evidence of severe hepatic impairment (Child-Pugh Class C)

7. Estimated GFR (eGFR) ≤30 mL/min/1.73 m² (based on CKD-EPI formula) at screening.

8. A stool analysis at screening with evidence of Clostridium difficile toxin, Salmonella, Shigella, Yersinia, Aeromonas, Plesiomonas Campylobacter or intestinal parasites

9. History of hypersensitivity or allergy to any component of the study drug.

10. Enrollment in another concurrent clinical interventional study, or intake of an investigational drug for COVID-19 within three months prior to randomization.

11. Foreseeable inability to cooperate with given instructions or study procedures.

### Patient Withdrawal

### Individual Treatment Discontinuation

The assigned study treatment might be permanently discontinued at any time by the investigator for safety or medically justified reasons.

If the assigned treatment is permanently discontinued the investigator needs to document and justify this in the e-CRF (electronic Case Report Form) as soon as possible.

Participant Withdrawal from the Study

A participant may withdraw the consent at any time for any reason.

In case of participant's permanent withdrawal, the investigator needs to document this in the e-CRF.

### Lost to Follow Up

A participant will be considered lost to follow-up if he/she fails provide data or biologic samples after the discharge at the time-points described in the protocol before the completion of the study.

The site should attempt to contact the patient (e.g. by phone or last known mailing address) in order to:

a) Determine if the patient is deceased and the reason of the death;

b) Reschedule, if still alive, the missed hospital or home visit as soon as possible and counsel the participant on the importance of maintaining the assigned visit schedule and ascertain whether the participant wishes to and/or should continue in the study.

Should the participant continue to be unreachable, he/she will be considered lost to follow up.

### Data Sources and Collection

Data required by the protocol will be collected both in hospital or at subjects' home, as applicable.

Data collection is the responsibility of the clinical study staff at the site, under the supervision of the Principal Investigator. The study eCRF is the primary data collection instrument for the study. The Investigator should ensure the accuracy, completeness, legibility, and timeliness of the data reported in the eCRFs and all other required reports.

The investigator must ensure that the clinical data required by the study protocol are carefully reported in the subject's source documents detailing the unique identification number and date and time of the study procedures performed. Data reported on the eCRF that are derived from source documents should be consistent with the source documents or the discrepancies should be explained. Any correction to the source data entries must be carried out by the investigator or a designated member of staff. Incorrect entries must not be covered with correcting fluid, or obliterated, or made illegible in any way.

All data requested on the eCRF must be recorded. Any missing data must be explained. An audit trail will be maintained by the eCRF system.

### Study Procedures

Potential study patients with confirmed long hauler's syndrome associated with COVID-19 diagnosis will be identified from those being treated at or referred to a participating clinical site. Clinical or laboratory procedures for the assessment of the inclusion and exclusion criteria will be performed after patient's signature of the informed consent. Each patient will be involved in the study for the entire duration of treatment either in hospital or at home and for the follow-up at home or in other institutions, if any, up to a maximum of 6 weeks.

The study flow chart of the study is shown in Table 15.

### Assessments

Assessments will be performed either at home or in an outpatient clinic. At-home assessments may be performed using a combination of remote digital monitoring of patient reported information, home visits by qualified research team staff (include nurses or medical assistants) and telehealth evaluation by study investigators. Implementation of the home-based outpatient assessment including remote digital monitoring and telehealth evaluation may include use a digital application (such as ApricityRx) for reporting daily health status and symptoms and use as a telemedicine interface configured for this study protocol.

### Assessments for Diarrhea

Assessments for the evaluation of diarrhea will be performed at all the study time-points and include:
Number of watery episodes per day according to Type 7 (liquid consistency with no solid pieces) of the Bristol Stool scale, as reported in Table 14 (Lewis et al., “Stool form scale as a useful guide to intestinal transit time”, Scand. J. Gastroentero. 32 (9): 920-4 (1997), which is hereby incorporated by reference in its entirety).
Number of evacuations with formed stool.
Use of anti-diarrheal agents.
Daily presence of other GI signs and symptoms: vomiting, nausea, anorexia, gastralgia, abdominal pain or discomfort, flatulence.

In the hospital the clinical assessment of diarrhea will be performed by the investigator, whereas after discharge it will be assessed daily and at defined study timepoints based on information entered by the patient using either a digital application or paper home diary.

TABLE 14
Bristol Stool Scale

| Type | Descriptor |
|---|---|
| Type 1 | Separate hard lumps, like nuts (difficult to pass and |
| | can be black) |
| Type 2 | Sausage-shaped, but lumpy |
| Type 3 | Like a sausage but with cracks on its surface (can be |
| | black) |
| Type 4 | Like a sausage or snake, smooth and soft (average stool) |
| Type 5 | Soft blobs with clear cut edges |
| Type 6 | Fluffy pieces with ragged edges, a mushy stool (diarrhea) |
| Type 7 | Watery, no solid pieces, entirely liquid (diarrhea) |

### Overall Clinical Assessment

The following information and clinical data will be captured as appropriate by the investigator, by the study nurse, or may be captured by a digital application daily and at defined study timepoints:
Death.
ICU admission.
Hospitalization.
WHO severity score as reported in Appendix C.
Duration, type of administration and quantity of supplemental oxygen treatment.
Blood oxygen saturation on room air (SaO2, if available, fingertip pulse oximeter).
Temperature including method of measurement (armpit, oral, rectal or tympanic).
Date of discharge or date of re-hospitalization.

### Drug Accountability

Investigational Medical Product (IMP) inventory and accountability records will be kept by the Investigator by means of a “IMP Accountability Log” and will include details of IMP received and a clear record of when it was dispensed and to which subjects. The Investigator will perform the drug accountability to calculate the number of tablets left. The Investigator agrees not to supply IMP to any person except subjects enrolled in this study

Safety and laboratory assessments
Safety (SAEs and AEs)
AST, ALT, LDH, gamma GT, total bilirubin and serum creatinine. All the other laboratory assessments are conducted according to the local practice and patient's clinical needs.

### Study Treatment

### Formulation and Packaging

The IMP is in the form of oral 400 mg uncoated tablets containing the active ingredient micronized niclosamide.

The chemical name of niclosamide is 5-chloro-N-(2-chloro-4-nitrophenyl)-2-hydroxybenzamide.

### Product Administration

Patients who are able to comply with the oral treatment, have to take, for each administration, one 400 mg tablet, for the total of three daily administrations, for 14 days. It is recommended to take the tablets after meals, with a glass of tap water to facilitate swallowing.

Management of vomiting after dose administration. If vomiting occurs within 1 hour after dosing, the dose should be re-administered. The re-administration should only occur once per planned dose; if vomiting occurs a second time after the same planned dose, it should not be administered a third time. The times and dose amount below are selected based on published gastric half-emptying times (Maes et al., “Pharmacological modulation of gastric emptying rate of solids as measured by the carbon labelled octanoic acid breath test: influence of erythromycin and propantheline”, Gut 35(3):333-7 (1994), which is hereby incorporated by reference in its entirety).

Diarrhea. No dose adjustment is required in the case of diarrhea as diarrhea primarily affects large intestinal transit which is less sensitive to transient dose effects (Read et al., “Transit of a meal through the stomach, small intestine, and colon in normal subjects and its role in the pathogenesis of diarrhea”, Gastroenterology 79(6):1276-1282 (1980), which is hereby incorporated by reference in its entirety).

Inability to tolerate oral tablet administration. For patients who are unable to tolerate or swallow tablets, it is possible based on the judgement of the investigator, to administer the IMP through a nasogastric tube dispersing, for each administration, one 400 mg tablet in 50 mL of drinking water in a suitable glass container and administering the mixture to the patient through a nasogastric tube using a needle-free syringe.

### Concomitant Medications and Treatments

In case of worsening or need of mechanical ventilation or admission to ICU patients will be treated with any medication or treatment based on the physicians' judgement, without any constraint from the sponsor, including but not limited to: antibiotics, antimycotics, steroids, alpha interferon, immunoglobulins.

After randomization and treatment allocation, the antiviral therapy can be modified by the investigator according to the patient's needs.

All concomitant medication and treatments taken or received during the study, from screening visit to discharge must be recorded. For concomitant or rescue medication: dose, posology, frequency of administration, start and end date and reason of use will be required and collected.

### Statistical Analysis Plan and Statistical Analysis

### Statistical Analysis Plan

All statistical methodology will be described in detail in the Statistical Analysis Plan (SAP) which will be finalized at the latest prior to database lock. All variables collected in the eCRF and/or other recordings (if applicable) and all derived parameters will be used in the statistical analysis.

### Sample Size Calculation

The sample size was determined by simulation. As described below, with a one-sided test for shorter time to clearance with niclosamide, a type-I error rate of 5%, and 50 patients per arm (100 patients total completing the study with an evaluable primary endpoint) the study is estimated to achieve approximately 96% power. In some scenarios 96% may appear to be over-powered, but, given the exploratory nature of the study and the unknown true effect size, the number of patients appears reasonable. The one-sided test was selected based on the fact that only the direction of shorter time to clearance may be relevant for improved clinical outcomes, and one-sided 5% significance (rather than 2.5%) was selected based on the fact that the trial is the first exploration of niclosamide treatment in this way and not a confirmatory, pivotal study.

The study is designed to achieve at least 90% power for detecting a centered 25% difference between the niclosamide and placebo arms in the exponential distribution rate associated with a 50% clearance of fecal SARS-CoV-2 samples 14 days after randomization (an average of 62.5% and 37.5% of subjects achieved viral clearance by that time for each arm, respectively). The simulations included rectal RT-qPCR viral detection with sampling scheduled on days 3, 7, 14, 21, 28, 35, and 42 after first dose; powering simulations did not include a requirement for replicated negative samples. The trial was simulated 10,000 times to assess the power.

### Analysis of Safety Variables

TEAEs, SAEs and AEs will be presented for each treatment arm in terms of number of AEs and their incidence by System Organ Class (SOC) and Preferred Terms (PT) using MedDRA. Analyses will be provided also by severity and relationship to the treatment.

Laboratory tests will be presented using descriptive statistics at each available visit.

Additionally, the frequency of subjects reporting an abnormal or abnormal clinically significant laboratory value at each available visit will be presented for each laboratory parameter.

### Interim Safety Analysis for Study Continue/Pause Decision

For the interim safety analysis, Fisher's exact test for count data will be used to compare the number of patients with and without either death or SAE. One test will be performed for death and one for SAE. The test will be a one-sided 95% confidence interval. If the lower bound of the confidence interval for active/placebo is >1, the study will be paused. If the lower bound of the confidence interval is ≤1, the study will continue. If one test indicates pause and the other indicates continue, the study will be paused. (For example, if the ratio of deaths indicates continue and the ratio of SAEs indicates pause, the study will pause).

In the case of a study pause, randomization will pause. If the lower bound of the confidence interval for active/placebo is >1 and ≤2, then dosing for patients currently in the study will continue. If the lower bound of the confidence interval for active/placebo is >2, then dosing for patients currently in the study will pause.

In the case of a study pause, patient data related to the SAEs or deaths will examine the reasons associated with each case, and if upon examination and concurrence with the IRB the imbalance is due to an external factor (e.g. an imbalance of severity of long hauler's syndrome associated with COVID-19 at randomization) then treatment may resume. If the imbalance is not due to an external factor or the IRB does not concur that the study may continue, the study will be stopped.

### Safety Reporting

### Definitions of Adverse Events and Serious Adverse Events

### Evaluation of Adverse Events

An AE is any untoward medical occurrence in a patient or clinical trial patient administered a pharmaceutical product and that does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product.

The period of observation for AEs extends from the time the patient gives informed consent until the trial is completed. AEs that are still present after the patient's last scheduled visit will be followed up by means of a phone call or visit, as considered appropriate. After that time point the need for additional follow-up of ongoing AEs/SAEs will be discussed between the investigator and the Sponsor, although in the event of discrepancies the investigator's criteria will prevail. AEs occurring after the end of the clinical trial must be reported if the investigator considers there is a causal relationship with the investigational product.

The investigator will be responsible for the necessary acute medical treatment of any AEs required during the trial and will ensure that appropriate medical care will be maintained thereafter, if necessary.

All patients experiencing AEs—whether considered related with the use of the IMP or not—will be monitored until symptoms subside and any abnormal laboratory values have returned to baseline, or until there is a satisfactory explanation for the changes observed, or until death, in which case a full pathologist's report will be supplied, if possible.

All AEs, including intercurrent illnesses, will be reported and documented as described below.

AEs are divided into the categories “serious” and “nonserious”. This determines the procedure which will be used to report/document the AE (see below).

Surgical procedures themselves are not AEs; they are therapeutic measures for conditions that require surgery. The condition for which the surgery is required is an AE, if it occurs or is detected during the trial period. Planned surgical measures permitted by the clinical trial protocol and the condition(s) leading to these measures are not AEs, if the condition(s) was (were) known before the start of treatment with investigational product. In the latter case the condition should be reported as medical history.

### Definition of Serious and Nonserious Adverse Events

A SAE is any untoward medical occurrence that at any dose:
results in death or is life-threatening;
results in permanent or significant disability/incapacity;
requires inpatient hospitalization or prolongation of hospitalization;
results in a congenital abnormality/birth defect;

Hospitalization solely for the purpose of diagnostic tests, even if related to an AE, elective hospitalization for an intervention which was already planned before the inclusion of the patient in the clinical trial and admission to a day-care facility may not themselves constitute sufficient grounds to be considered as a SAE.

Medical and scientific judgment will be exercised in classification of other important medical events that may not be immediately life-threatening or result in death or hospitalization but may jeopardize the patient or may require intervention to prevent one of the other outcomes listed in the definition above. These should also usually be considered serious.

AEs which do not fall into these categories are defined as nonserious.

### Reporting/Documentation of Adverse Events

The investigator or qualified sub-investigator is responsible for assessing AEs and SAES for causality and severity, and for final review and confirmation of accuracy of event information and assessments.

AEs either reported by the patient, or observed by the investigator must be recorded on the AE section of the eCRF and should be described in the following manner:

The nature of the event will be described in precise, standard medical terminology. If known, a specific diagnosis should be stated (e.g., allergic contact dermatitis).

The severity of the AE will be described in terms of mild, moderate or severe according to the investigator's clinical judgment.
Mild: The AE does not interfere in a significant manner with the patient's normal functioning level, but may be an annoyance.
Moderate: The AE produces some impairment of functioning but is not hazardous to health, but is uncomfortable and/or an embarrassment.
Severe: The AE produces significant impairment of functioning or incapacitation and is a hazard to the patient.

The duration of the event will be described by the start date and end date.

The location for cutaneous AEs will be described as at or just around the application area (≤2 cm from the application area) or distant (>2 cm from the application area).

The causal relationship of the event to the use of the IMP the investigator or qualified sub-investigator is responsible for assessing the relationship to study drug using clinical judgment and the following considerations:
Certain: the AE occurs in a plausible time relationship to IMP administration, and cannot be explained by concurrent disease or other drugs or chemicals, and follows a clinically plausible response to withdrawal of the IMP, and is definitive based on recognized pharmacological or other parameter associated with the IMP, and is confirmed by re-challenge procedure, if performed.
Probable: the AE follows a reasonable temporal sequence from administration of the IMP, and is unlikely to be attributed to a disease or other drug/s, and disappears or decreases on withdrawal of the IMP
Possible: the AE follows a reasonable temporal sequence from administration of the IMP but can also be explained by disease or other drugs, and information on drug withdrawal may be lacking or unclear.
Unlikely: the AE does not follow a reasonable temporal sequence from administration of the IMP and can be reasonably explained by disease or other drug/s, and does not follow a known pattern of response to the IMP, and does not reappear or worsen upon re-challenge, if performed.
Not related: the AE occurs prior to IMP administration.

The outcome of the event will be described in terms of (a) Recovered/resolved; (b) Recovering/resolving; (c) Recovered/resolved with sequelae; (d) Not recovered/not resolved; (e) Fatal; (f) Unknown. It will also be recorded if the study product use is continued, interrupted or discontinued.

1. A method of treating long-haulers syndrome associated with Covid-19 infection comprising administering a therapeutically effective amount of niclosamide or a pharmaceutically acceptable salt thereof to a patient in need thereof without the concomitant administration of a protease inhibitor.

2. The method of claim 1, wherein the administration treats symptoms associated with one or more organ systems selected from gastrointestinal, pulmonary, renal, hepatic, dermatologic, coronary and nervous.

3. The method of claim 2, wherein the symptoms of the organ systems are associated with one or more organs selected from intestine, lungs, heart and brain.

4. The method of any preceding claims, wherein the treatment provides one or both of an anti-inflammatory response and an antiviral response.

5. The method of claim 4, wherein the administration is in an effective amount to cause an antiviral response by induction of autophagy.

6. The method of claim 4, wherein the administration is in an effective amount to cause an anti-inflammatory response by one or more of mitigation of pathogenic lamina propria T cells or reduction of pro-inflammatory cytokines.

7-8. (canceled)

9. The method of claim 1, wherein administration is by a route selected from oral, nasal, inhalation, rectal, topical, parenteral or transdermal.

10. The method of claim 9, wherein the administration is by inhalation.

11. The method of claim 10, wherein the administration is by dry powder, aerosols, nebulization or intubation.

12. The method of claim 9, wherein the administration is rectal.

13. The method of claim 12, wherein the administration is by enema or suppository.

14. The method of claim 9, wherein the administration is oral.

15. (canceled)

16. The method of claim 1, wherein a daily dose is from about 200 mg to about 1800 mg.

17. The method of claim 1, wherein a daily dose is administered once daily.

18. The method of claim 1, wherein a daily dose is administered in divided doses twice daily, thrice daily, four times daily, 5 times daily or 6 times daily.

19. The method of claim 1, wherein the niclosamide or pharmaceutically acceptable salt thereof is administered 400 mg three times daily.

20. The method of claim 14, wherein the oral administration is with an oral solid dosage form.

21. The method of claim 20, wherein the oral solid dosage form is a tablet or capsule.

22. The method of claim 21, wherein the tablet or capsule comprises an enteric coating.

23. The method of claim 22, wherein the enteric coating is an acrylic polymer or a cellulosic phthalate.

24. The method of claim 14, wherein the oral administration is with an immediate release dosage form, a controlled release dosage form, a delayed release dosage form, a sustained release dosage form, a pulsatile dosage form or a chronotherapeutic dosage form.

25. The method of claim 14, wherein the administration is with food.

26-35. (canceled)

36. The method of claim 1, wherein the niclosamide or pharmaceutically acceptable salt thereof is micronized.

37. The method of claim 36, wherein the D90 of the niclosamide or pharmaceutically acceptable salt thereof is from about 1 micron to about 20 microns.

38. The method of claim 37, wherein the administration results in a concentration of niclosamide in the intestine which is more than the systemic plasma.

39. The method of claim 38, wherein the ratio of the concentration of niclosamide in the intestine to the systemic plasma is greater than 100.

40. The method of claim 1, further comprising administering an anti-viral agent, an antibiotic or an antiparasitic.

41. The method of claim 40, wherein the anti-viral agent is a viral mimetic, a nucleotide analog, a nucleotide mimic, a sialidase inhibitor, or a protease inhibitor.

42-49. (canceled)

50. The method of claim 1, wherein the administration treats one or more symptoms selected from fatigue, shortness of breath, cough, joint pain, chest pain, difficulty with thinking and concentration (brain fog), depression, muscle pain, headache, intermittent fever, heart palpitations, inflammation of the heart muscle, lung function abnormalities, acute kidney injury, rash, hair loss, smell and taste problems, sleep issues, difficulty with concentration, memory problems, depression, anxiety and change in mood.

51-62. (canceled)

63. A method of providing prophylaxis of long hauler's syndrome associated with Covid-19 infection comprising administering a therapeutically effective amount of niclosamide or a pharmaceutically acceptable salt thereof to a patient in need thereof without the concomitant administration of a protease inhibitor.
